(19) United States
(12) Patent Application Publication
(10) Pub. No.: US 2026/0108738 A1
(43) Pub. Date: Apr. 23, 2026

(54) REDUCING STIMULATION ARTIFACTS IN COCHLEAR IMPLANT SYSTEMS

(71) Applicant: Envoy Medical Corporation, White Bear Lake, MN (US)

(72) Inventors: Thomas B. Hoegh, Edina, MN (US); Kevin Richard Vold, Minneapolis, MN (US)

(21) Appl. No.: 19/360,839

(22) Filed: Oct. 16, 2025

Related U.S. Application Data
(60) Provisional application No. 63/708,428, filed on Oct. 17, 2024.

(51) Int. Cl. A61N 1/36; A61N 1/05; A61N 1/375

(52) U.S. Cl. CPC A61N 1/36038; A61N 1/0541; A61N 1/36125; A61N 1/36153; A61N 1/36157; A61N 1/36171; A61N 1/36185; A61N 1/3754

(57) ABSTRACT

A cochlear implant system can reduce noise in an input signal from a sensor due to artifacts from electrical stimulation. A balancing capacitance can be coupled between a signal-carrying conductor and a reference voltage or a portion of an implantable housing to balance a mismatch in capacitive coupling of the interference to different conductors to facilitate reduction of the artifact. An input signal representative of a received acoustic stimulus can be filtered to attenuate one or more frequencies to reduce the stimulation artifact. Frequencies near those attenuated can be amplified to compensate for any useful signal reduced by the attenuation. Stimulation artifacts can be estimated and subtracted from an input signal. Stimulation artifacts can be estimated as a combination basis functions of artifact contributions from a plurality of stimulation electrodes.

### CROSS-REFERENCE

This application claims priority to U.S. Provisional Patent Application No. 63/708,428, filed Oct. 17, 2024, the entire contents of which are incorporated herein by reference.

### TECHNICAL FIELD

Aspects of this disclosure relate to cochlear implant systems.

### BACKGROUND

A cochlear implant is an electronic device that may be at least partially implanted surgically into the cochlea, the hearing organ of the inner ear, to provide improved hearing to a patient. Cochlear implants may include components that are worn externally by the patient and components that are implanted internally in the patient.

Cochlear implant systems can provide improved hearing to a patient by receiving external stimuli and generating stimulation signals, such as electrical signals, based on the received external stimuli. However, noise or interference present at any stage of the implant system can negatively impact the operation of the system, for example, by reducing the accuracy of the representation of the external stimuli by the stimulation signals.

### SUMMARY

Some aspects of this disclosure are related to cochlear implant systems. Systems can include an input source configured to receive an acoustic stimulus and generate an input signal representative of the acoustic stimulus. Systems can include a cochlear electrode comprising a plurality of contact electrodes and a stimulator in communication with the cochlear electrode and configured to provide electrical stimulation to cochlear tissue via the contact electrodes of the cochlear electrode. Systems can further include a signal processor in communication with the stimulator and the input source. The signal processor can be programmed with a transfer function and configured to receive the input signal from the input source at a signal processor input and generate a stimulation signal based on the received input signal and a transfer function of the signal processor. The stimulation signal can include a stimulation pattern having an electrical output corresponding to each of the plurality of contact electrodes.

In some examples, interference can be introduced into the system due to electrical signals present within the system. For instance, in some examples, electrical signals associated with electrical stimulation can introduce a stimulation artifact that can cause electrical interference at the signal processor input, which can affect the input signal processed by the signal processor and the resulting electrical stimulation. Accordingly, in some examples, interference from the stimulation artifact associated with electrical stimulation can introduce noise into an input signal received by the signal processor, and that noise, if unaddressed, can manifest in noise in the stimulation provided to a wearer. In some cases, interference has been found to be present when voltage is present at one or more contact electrodes independent of whether current is being provided from the contact electrode(s).

Some aspects of this disclosure are related to addressing interference associated with electrical stimulation. In some cases, electrical interference associated with stimulation can be measured or otherwise determined (e.g., by way of a lookup table linking interference with certain stimulation parameters, such as voltage, current, or impedance) and compensated for, for example, by subtracting a measured or otherwise determined interference from the input signal by the signal processor.

Filters can be used to attenuate stimulation artifacts within an input signal. In some examples, one or more frequencies associated with stimulation (e.g., a stimulation frame rate) can be attenuated to reduce stimulation artifacts having such frequency content. In some examples, one or more frequencies or ranges of frequencies of an input signal can be attenuated to reduce stimulation artifacts in the input signal.

In some embodiments, a cochlear implant system can include an input source configured to receive an acoustic stimulus and generate an input signal representative of the acoustic stimulus, a stimulator configured to provide electrical stimulation to cochlear tissue via a cochlear electrode, and a signal processor in communication with the stimulator and the input source. The signal processor can be programmed with a transfer function and be configured to receive the input signal, decompose the input signal into a plurality of frequency bands, apply a filter to the input signal to attenuate one or more frequencies associated with electrical stimulation, and amplify the input signal within a frequency band that includes one or more frequencies attenuated by the filter.

In some embodiments, the filter applied by the signal processor is configured to attenuate frequencies associated with a stimulation frame rate of the electrical stimulation, and may further attenuate one or more harmonics of the stimulation frame rate. The filter may be implemented as a comb filter to attenuate a plurality of discrete frequencies or narrow frequency ranges within the input signal. The signal processor can determine which of the plurality of frequency bands include attenuated frequencies and selectively amplify the input signal within those bands to compensate for the attenuation, thereby preserving the overall amplitude and fidelity of the input signal used for generating stimulation.

Additionally, the signal processor may be configured to determine an amount of amplification based on the amount of attenuation within a frequency band and to amplify frequencies within that band by the determined amount. In some embodiments, the signal processor can also amplify the input signal within a second frequency band, separate from the first frequency band, in response to attenuation of a frequency within the second frequency band by the filter. These features enable the cochlear implant system to reduce the impact of stimulation artifacts while maintaining the integrity of the acoustic information delivered to the user.

In some cases, stimulation artifacts causing interference are a sum of individual interference contributions from individual contact electrodes. Basis functions can be determined for each contact electrode in order to estimate, for example, as a function of stimulation output and/or anatomical parameters, a contribution from each contact electrode to the overall stimulation artifact. A cochlear implant system (e.g., via an implantable signal processor) can be configured to use such basis functions in combination with output stimulation in order to estimate an overall stimulation artifact due to the contributions from each contact electrode providing stimulation and subtract the estimated artifact from the signal received from the sensor in order to generate a modified input signal for further processing and generating stimulation.

The stimulation artifact can act as an aggressor signal capacitively coupling to aspects of the cochlear implant system, such as a lead connecting an input source and a housing enclosing a signal processor and/or a header of the housing. Capacitive mismatches between the coupling of the aggressor signal to different conductors can cause the aggressor signal to be amplified by one or more differential amplifiers within a signal path. Balancing the capacitive mismatches can cause the aggressor signal to couple equally to both inputs to a differential amplifier such that the aggressor signal is eliminated in the differential amplification.

Additionally or alternatively, a pre-amplifier can be used to amplify the signal from the sensor in order to improve the signal-to-noise ratio of the signal from the sensor compared to the aggressor. Pre-amplifiers can be combined with capacitive balancing to further mitigate the impact of the stimulation artifact on signals to be processed to generate stimulation signals.

Various combinations of such stimulation artifact reduction or elimination techniques can be performed to improve the fidelity of signals used for generating stimulation relative to detected acoustic stimuli that the stimulation is intended to represent.

### BRIEF DESCRIPTION OF THE DRAWINGS

FIG. 1 shows an illustration of an example fully implantable cochlear implant system.

FIG. 2 shows an embodiment of a fully implantable cochlear implant.

FIG. 3 shows an embodiment of an example middle ear sensor for use in conjunction with anatomical features of a patient.

FIG. 4 shows a diagram illustrating an example implantable system including an acoustic stimulator.

FIG. 5A shows an example illustration of processor and stimulator combined into a single housing.

FIG. 5B shows a simplified cross-sectional view of the processor/stimulator shown in FIG. 5A.

FIG. 6 shows an example cochlear electrode comprising a plurality of contact electrodes.

FIG. 7 shows an example diagram illustrating various system components including a signal processor.

FIG. 8 shows a transform (e.g., FFT) of an input signal showing frequency content of interference.

FIG. 9 shows an example process of addressing interference by modifying an input signal.

FIG. 10 shows an example process of addressing interference by updating a signal processor transfer function.

FIG. 11 shows an example process of addressing interference by modifying an input signal and updating a signal processor transfer function.

FIG. 12A shows an example signal from an input source while the system outputs a stimulation pattern at a stimulation frame rate.

FIG. 12B shows a zoomed-in section of the signal in FIG. 12A.

FIG. 12C shows 4096 data points repeatedly overlaid on a single time period of one stimulation frame.

FIG. 13 shows an illustration of an example adaptive filter.

FIG. 14A shows a desired signal prior to the addition of a stimulation artifact thereto in the form of a pure 1 kHz sine wave.

FIG. 14B shows a combined signal, which includes a stimulation artifact combined into the desired signal of FIG. 14A.

FIG. 14C shows the signal resulting from applying the time domain adaptive filter to the signal of FIG. 14B.

FIG. 14D shows the initial signal of FIG. 14A overlaid onto the result of the filter shown in FIG. 14C.

FIG. 15A shows an example circuit diagram sensor assembly comprising a sensor outputting a signal affected by an aggressor signal.

FIG. 15B shows an example circuit diagram showing the effect of a capacitive coupling mismatch of an aggressor signal on a differential amplifier.

FIG. 15C shows an example network of electrical components for processing differential signals from a sensor assembly.

FIG. 15D shows example cochlear implant system components that can be used in some embodiments to provide electrical stimulation based on signals from the sensor.

FIG. 16A shows an example low side balanced network approach to addressing mismatched capacitive coupling of an aggressor signal.

FIG. 16B shows a circuit diagram illustrating the effect of a low side balancing capacitance on capacitive coupling of the aggressor signal.

FIG. 17A shows an example high side balanced network approach to addressing mismatched capacitive coupling of an aggressor signal.

FIG. 17B shows a circuit diagram illustrating the effect of high side balancing capacitance on capacitive coupling of the aggressor signal.

FIG. 18A shows an example sensor assembly circuit with a single-ended pre-amplifier and capacitive coupling of an aggressor signal.

FIG. 18B shows an example circuit diagram of a single-ended pre-amplifier amplifying a sensor signal for input to a differential amplifier without an aggressor signal.

FIG. 18C shows an example illustration of an aggressor signal coupling to signals from a single-ended pre-amplifier, with a negative amplified signal being grounded.

FIG. 19A shows an example configuration of a sensor assembly comprising a sensor and a differential pre-amplifier and capacitive coupling of an aggressor signal.

FIG. 19B shows a circuit diagram of a differential pre-amplifier amplifying a sensor signal for input to a differential amplifier without an aggressor signal.

FIG. 19C shows an example illustration of an aggressor signal coupling to amplified signals from a differential pre-amplifier.

FIG. 20 shows a sensor assembly circuit incorporating a differential pre-amplifier and high side balancing capacitance.

FIG. 21 shows a process flow diagram showing an example process for establishing basis functions for one or more contact electrodes and stimulation artifact reduction in a cochlear implant system.

FIG. 22 shows a process flow diagram showing example operation of a cochlear implant system reducing stimulation artifacts using basis functions.

FIG. 23 shows a process flow diagram illustrating an example process of attenuating one or more frequencies from an input signal and amplifying portions of the input signal to compensate for signal lost by the attenuation.

### DETAILED DESCRIPTION

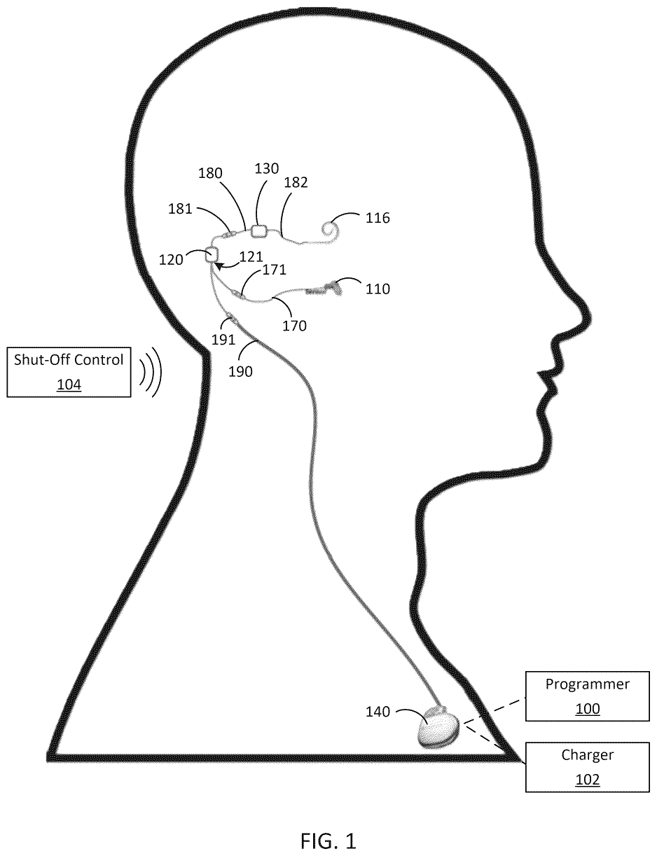

FIG. 1 shows an illustration of an example fully implantable cochlear implant system. The system of FIG. 1 includes a middle ear sensor 110 in communication with a signal processor 120. The middle ear sensor 110 can be configured to detect incoming sound waves, for example, using the ear structure of a patient. While some embodiments are described as including a middle ear sensor, in various examples, one or more additional or alternative input sources, such as an implantable microphone or other source, can be configured to detect incoming sound waves. The signal processor 120 can be configured to receive a signal from the middle ear sensor 110 or other input source and produce an output signal based thereon. For example, the signal processor 120 can be programmed with instructions to output a certain signal based on a received signal. In some embodiments, the output of the signal processor 120 can be calculated using an equation based on received input signals. Alternatively, in some embodiments, the output of the signal processor 120 can be based on a lookup table or other programmed (e.g., in memory) correspondence between the input signal from the middle ear sensor 110 and the output signal. While not necessarily based explicitly on a function, the relationship between the input to the signal processor 120 (e.g., from the middle ear sensor 110) and the output of the signal processor 120 is referred to as the transfer function of the signal processor 120.

In various examples, the signal processor 120 can comprise any variety of components, for example, digital and/or analog processing components. In some embodiments, signal processor 120 comprises a digital signal processor, one or more microprocessors, microcontrollers, application specific integrated circuits (ASICs) or the like. Supporting circuitry for one or more such components can also be included as a part of the signal processor. In some embodiments, the signal processor can include or otherwise communicate with a memory containing programming for operating one or more components. Additionally or alternatively, in some embodiments, the signal processor can include one or more additional components. For example, in some embodiments, signal processor can include an embedded microphone or other sensor configured to detect incoming sound waves.

The system of FIG. 1 further includes a cochlear electrode 116 implanted into the cochlear tissues of a patient. The cochlear electrode 116 is in electrical communication with an electrical stimulator 130, which can be configured to provide electrical signals to the cochlear electrode 116 in response to input signals received by the electrical stimulator 130. In some examples, the cochlear electrode 116 is fixedly attached to the electrical stimulator 130. In other examples, the cochlear electrode 116 is removably attached to the electrical stimulator 130. As shown, the electrical stimulator 130 is in communication with the signal processor 120. In some embodiments, the electrical stimulator 130 provides electrical signals to the cochlear electrode 116 based on signals received from the signal processor 120. For instance, in some examples, the signal processor 120 is configured to receive an input signal from an input source, such as a middle ear sensor 110, and generate a stimulation signal for outputting to the electrical stimulator 130. The electrical stimulator can be configured to provide electrical stimulation via cochlear electrode 116 based on the stimulation signal received from the signal processor 120.

In various embodiments, the cochlear electrode 116 can include any number of contact electrodes in electrical contact with different parts of the cochlear tissue. In such embodiments, the electrical stimulator 130 can be configured to provide electrical signals to any number of such contact electrodes to stimulate the cochlear tissue. For example, in some embodiments, the electrical stimulator 130 is configured to activate different contact electrodes or combinations of contact electrodes of the cochlear electrode 116 in response to different input signals received from the signal processor 120. This can help the patient differentiate between different input signals.

During example operation, the middle ear sensor 110 detects audio signals, for example, using features of the patient's ear anatomy as described elsewhere herein and in U.S. Pre-Grant Patent Publication No. 2013/0018216, which is hereby incorporated by reference in its entirety. The signal processor 120 can receive such signals from the middle ear sensor 110 and produce an output to the electrical stimulator 130 based on the transfer function of the signal processor 120. The electrical stimulator 130 can then stimulate one or more contact electrodes of the cochlear electrode 116 based on the received signals from the signal processor 120.

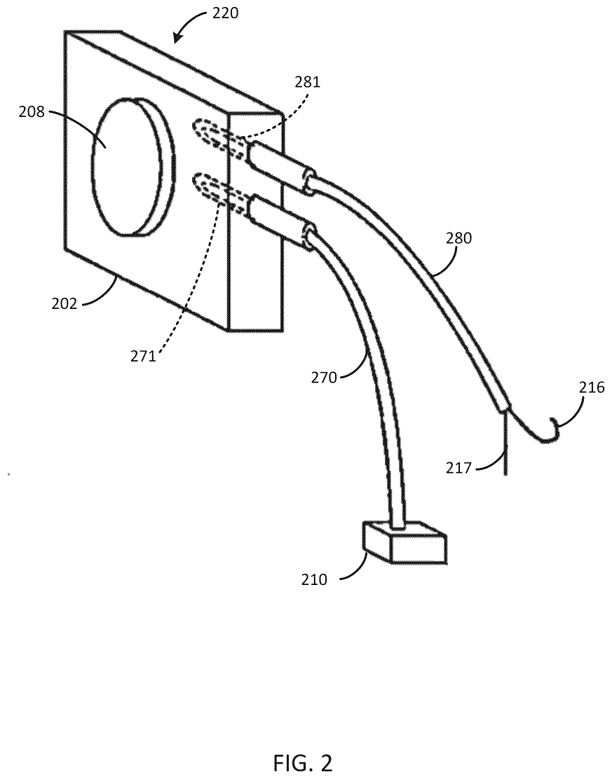

Referring to FIG. 2, an embodiment of a fully-implantable cochlear implant is shown. The device in this embodiment includes a processor 220 (e.g., signal processor), a sensor 210, a first lead 270 connecting the sensor 210 to the processor 220, and a combination lead 280 attached to the processor 220, wherein combination lead 280 contains both a ground electrode 217 and a cochlear electrode 216. The illustrated processor 220 includes a housing 202, a coil 208, first female receptacle 271 and second female receptacle 281 for insertion of the leads 270 and 280, respectively.

In some embodiments, coil 208 can receive power and/or data from an external device, for instance, including a transmission coil (not shown). Some such examples are described in U.S. Pre-Grant Patent Publication No. 2013/0018216, which is incorporated by reference. In other examples, processor 220 is configured to receive power and/or data from other sources, such as an implantable battery and/or communication module as shown in FIG. 1. Such battery and/or communication module can be implanted, for example, into the pectoral region of the patient in order to provide adequate room for larger equipment (e.g., a relatively large battery) for prolonged operation (e.g., longer battery life). Additionally, in the event a battery needs eventual replacement, a replacement procedure in the patient's pectoral region can be performed several times without certain vascularization issues that can arise near the location of the cochlear implant. For example, in some cases, repeated procedures (e.g., battery replacement) near the cochlear implant can result in a decreased ability for the skin in the region to heal after a procedure. Placing a replaceable component such as a battery in the pectoral region can facilitate replacement procedures with reduced risk for such issues.

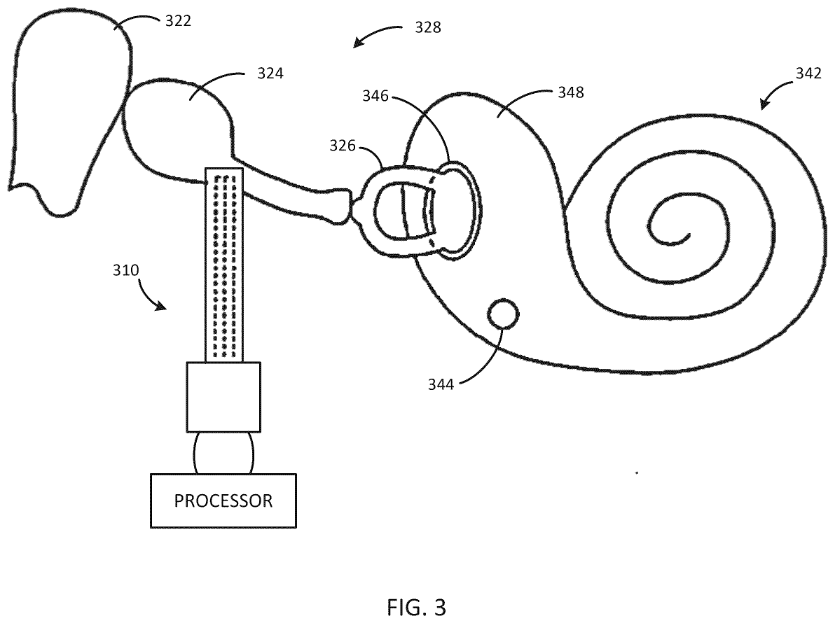

FIG. 3 illustrates embodiments of an example middle ear sensor for use in conjunction with anatomical features of a patient. Referring to FIG. 3, an embodiment of the sensor 310 of a fully-implantable cochlear implant is shown. Also shown are portions of the subject's anatomy, which includes, if the subject is anatomically normal, at least the malleus 322, incus 324, and stapes 326 of the middle ear 328, and the cochlea 348, oval window 346, and round window 344 of the inner ear 342. Here, the sensor 310 is touching the incus 324. The sensor 310 can include a sensor such as described in U.S. Pre-Grant Patent Publication No. 2013/0018216, which is incorporated by reference. Further, although not shown in a drawing, the sensor 310 may be in operative contact with the tympanic membrane or the stapes, or any combination of the tympanic membrane, malleus 322, incus 324, or stapes 326.

FIG. 3 illustrates an example middle ear sensor for use with systems described herein. However, other middle ear sensors can be used, such as sensors using microphones or other sensors capable of receiving an input corresponding to detected sound and outputting a corresponding signal to the signal processor. Additionally or alternatively, systems can include other sensors configured to output a signal representative of sound received at or near a user's ear, such as a microphone or other acoustic pickup located in the user's outer ear or implanted under the user's skin. Such devices may function as an input source, for example, to the signal processor such that the signal processor receives an input signal from the input source and generates and outputs one or more stimulation signals according to the received input signal and the signal processor transfer function. Additionally or alternatively, systems can include other types of sensors, such as inner ear sensors. Some example configurations of such systems and other sensor arrangements are described in U.S. Pat. No. 11,260,220, entitled IMPLANTABLE COCHLEAR SYSTEM WITH INTEGRATED COMPONENTS AND LEAD CHARACTERIZATION, and which is assigned to the assignee of the instant application and is incorporated by reference.

Referring back to FIG. 1, the signal processor 120 is shown as being in communication with the middle ear sensor 110, the electrical stimulator 130, and the implantable battery and/or communication module 140. As described elsewhere herein, the signal processor 120 can receive input signals from the middle ear sensor 110 and/or other input source(s) and output signals to the electrical stimulator 130 for stimulating the cochlear electrode 116. The signal processor 120 can receive data (e.g., processing data establishing or updating the transfer function of the signal processor 120) and/or power from the implantable battery and/or communication module 140.

In some embodiments, the implantable battery and/or communication module 140 can communicate with one or more external components, such as a programmer 100 and/or a battery charger 102. The battery charger 102 can wirelessly charge the battery in the implantable battery and/or communication module 140 when brought into proximity with the implantable battery and/or communication module 140 in the pectoral region of the patient. Such charging can be accomplished, for example, using inductive charging. The programmer 100 can be configured to wirelessly communicate with the implantable battery and/or communication module 140 via any appropriate wireless communication technology, such as Bluetooth, Wi-Fi, and the like. In some examples, the programmer 100 can be used to update the system firmware and/or software. In an example operation, the programmer 100 can be used to communicate an updated signal processor 120 transfer function to the implantable battery and/or communication module 140. In various embodiments, the programmer 100 and battery charger 102 can be separate devices or can be integrated into a single device.

In the illustrated example of FIG. 1, the signal processor 120 is connected to the middle ear sensor 110 via lead 170. In some embodiments, the signal processor 120 comprises a signal processor input 121 such that the signal processor 120 is configured to receive an input signal from an input source (e.g., middle ear sensor 110) at the signal processor input 121. In some such examples, the signal processor input 121 can be configured to interface with lead 170.

While the example shown in FIG. 1 illustrates leads 170 and 190 interfacing with the signal processor 120 at a single input (e.g., signal processor input 121), in various embodiments, the signal processor 120 interfaces with leads 170 and 190 at separate locations, and such locations can be on a same side of the signal processor 120 or on different sides of the signal processor.

In some embodiments, lead 170 can provide communication between the signal processor 120 and the middle ear sensor 110. In some embodiments, lead 170 can include a plurality of isolated conductors providing a plurality of communication channels between the middle ear sensor 110 and the signal processor 120. The lead 170 can include a coating such as an electrically insulating sheath to minimize any conduction of electrical signals to the body of the patient. In various embodiments, one or more communication leads can be detachable such that communication between two components can be disconnected in order to electrically and/or mechanically separate such components. For instance, in some embodiments, lead 170 includes a detachable connector 171. Detachable connector 171 can facilitate decoupling of the signal processor 120 and middle ear sensor 110. Example detachable connectors are described in U.S. Pat. No. 11,260,220, which is incorporated by reference. For example, with reference to FIG. 1, in some embodiments, lead 170 can include a first lead extending from the middle ear sensor 110 having one of a male or a female connector and a second lead extending from the signal processor 120 having the other of the male or female connector. The first and second leads can be connected at detachable connector 171 in order to facilitate communication between the middle ear sensor 110 and the signal processor 120.

In other examples, a part of the detachable connector 171 can be integrated into one of the middle ear sensor 110 and the signal processor 120. For example, in an example embodiment, the signal processor 120 can include a female connector integrated into a housing of the signal processor 120. Lead 170 can extend fully from the middle ear sensor 110 and terminate at a corresponding male connector for inserting into the female connector of the signal processor 120. In still further embodiments, a lead (e.g., 170) can include connectors on each end configured to detachably connect with connectors integrated into each of the components in communication. For example, lead 170 can include two male connectors, two female connectors, or one male and one female connector for detachably connecting with corresponding connectors integral to the middle ear sensor 110 and the signal processor 120. Thus, lead 170 may include two or more detachable connectors.

Similar communication configurations can be established for detachable connector 181 of lead 180 facilitating communication between the signal processor 120 and the stimulator 130 and for detachable connector 191 of lead 190 facilitating communication between the signal processor 120 and the implantable battery and/or communication module 140. Leads (170, 180, 190) can include pairs of leads having corresponding connectors extending from each piece of communicating equipment, or connectors can be built in to any one or more communicating components.

In such configurations, each of the electrical stimulator 130, signal processor 120, middle ear sensor 110, and battery and/or communication module can each be enclosed in a housing, such as a hermetically sealed housing comprising biocompatible materials. Such components can include feedthroughs providing communication to internal components enclosed in the housing. Feedthroughs can provide electrical communication to the component via leads extending from the housing and/or connectors integrated into the components. For instance, in some embodiments, a signal processor input (e.g., 121) comprises a feedthrough such that the signal processor 120 is configured to receive an input signal from an input source (e.g., middle ear sensor) via a lead (e.g., 170) coupled to the feedthrough of the signal processor input 121. In some embodiments, one or more feedthroughs each comprise a pin at a location outside of a component (e.g., outside of a signal processor input 121) and providing electrical communication into an interior of the component (e.g., to one or more components within a signal processor housing).

In a module configuration such as that shown in FIG. 1, various components can be accessed (e.g., for upgrades, repair, replacement, etc.) individually from other components. For example, as signal processor 120 technology improves (e.g., improvements in size, processing speed, power consumption, etc.), the signal processor 120 implanted as part of the system can be removed and replaced independently of other components. In an example procedure, an implanted signal processor 120 can be disconnected from the electrical stimulator 130 by disconnecting detachable connector 181, from the middle ear sensor 110 by disconnecting detachable connector 171, and from the implantable battery and/or communication module 140 by disconnecting detachable connector 191. Thus, the signal processor 120 can be removed from the patient while other components such as the electrical stimulator 130, cochlear electrode 116, middle ear sensor 110, and battery and/or communication module can remain in place in the patient.

After the old signal processor is removed, a new signal processor can be connected to the electrical stimulator 130, middle ear sensor 110, and implantable battery and/or communication module 140 via detachable connectors 181, 171, and 191, respectively. Thus, the signal processor (e.g., 120) can be replaced, repaired, upgraded, or any combination thereof, without affecting the other system components. This can reduce, among other things, the risk, complexity, duration, and recovery time of such a procedure. In particular, the cochlear electrode 116 can be left in place in the patient's cochlea while other system components can be adjusted, reducing trauma to the patient's cochlear tissue.

Such modularity of system components can be particularly advantageous when replacing a signal processor 120, such as described above. Processor technology continues to improve and will likely continue to markedly improve in the future, making the signal processor 120 a likely candidate for significant upgrades and/or replacement during the patient's lifetime. Additionally, in embodiments such as the embodiment shown in FIG. 1, the signal processor 120 communicates with many system components. For example, as shown, the signal processor 120 is in communication with each of the electrical stimulator 130, the middle ear sensor 110, and the implantable battery and/or communication module 140. Detachably connecting such components with the signal processor 120 (e.g., via detachable connectors 181, 171, and 191) enables replacement of the signal processor 120 without disturbing any other components. Thus, in the event of an available signal processor 120 upgrade and/or a failure of the signal processor 120, the signal processor 120 can be disconnected from other system components and removed.

While many advantages exist for a replaceable signal processor 120, the modularity of other system components can be similarly advantageous, for example, for upgrading any system component. Similarly, if a system component (e.g., the middle ear sensor 110) should fail, the component can be disconnected from the rest of the system (e.g., via detachable connector 171) and replaced without disturbing the remaining system components. In another example, even a rechargeable battery included in the implantable battery and/or communication module 140 may eventually wear out and need replacement. The implantable battery and/or communication module 140 can be replaced or accessed (e.g., for replacing the battery) without disturbing other system components. Further, as discussed elsewhere herein, when the implantable battery and/or communication module 140 is implanted in the pectoral region of the patient, such as in the illustrated example, such a procedure can leave the patient's head untouched, eliminating unnecessarily frequent access beneath the skin.

While various components are described herein as being detachable, in various embodiments, one or more components configured to communicate with one another can be integrated into a single housing. For example, in some embodiments, signal processor 120 can be integrally formed with the stimulator 130 and cochlear electrode 116. For example, in an example embodiment, processing and stimulation circuitry of a signal processor 120 and stimulator 130 can be integrally formed as a single unit in a housing coupled to a cochlear electrode. Cochlear electrode and the signal processor/stimulator can be implanted during an initial procedure and operate as a single unit.

In some embodiments, while the integral signal processor/stimulator/cochlear electrode component does not get removed from a patient due to potential damage to the cochlear tissue into which the cochlear electrode is implanted, system upgrades are still possible. For example, in some embodiments, a modular signal processor may be implanted alongside the integral signal processor/stimulator component and communicate therewith. In some such examples, the integral signal processor may include a built-in bypass to allow a later-implanted signal processor to interface directly with the stimulator. Additionally or alternatively, the modular signal processor can communicate with the integral signal processor, which may be programmed with a unity transfer function. Thus, in some such embodiments, signals from the modular signal processor may be essentially passed through the integral signal processor unchanged so that the modular signal processor effectively controls action of the integral stimulator. Thus, in various embodiments, hardware and/or software solutions exist for upgrading an integrally attached signal processor that may be difficult or dangerous to remove.

While often described herein as using an electrical stimulator to stimulate the patient's cochlear tissue via a cochlear electrode, in some examples, the system can additionally or alternatively include an acoustic stimulator. An acoustic stimulator can include, for example, a transducer (e.g., a piezoelectric transducer) configured to provide mechanical stimulation to the patient's ear structure. In an example embodiment, the acoustic stimulator can be configured to stimulate one or more portions of the patient's ossicular chain via amplified vibrations. Acoustic stimulators can include any appropriate acoustic stimulators, such as those found in the ESTEEM™ implant (Envoy Medical Corp.) or as described in U.S. Pat. Nos. 4,729,366, 4,850,962, and 7,524,278, and U.S. Pre-Grant Patent Publication No. 2010/0042183, each of which is incorporated herein by reference in its entirety.

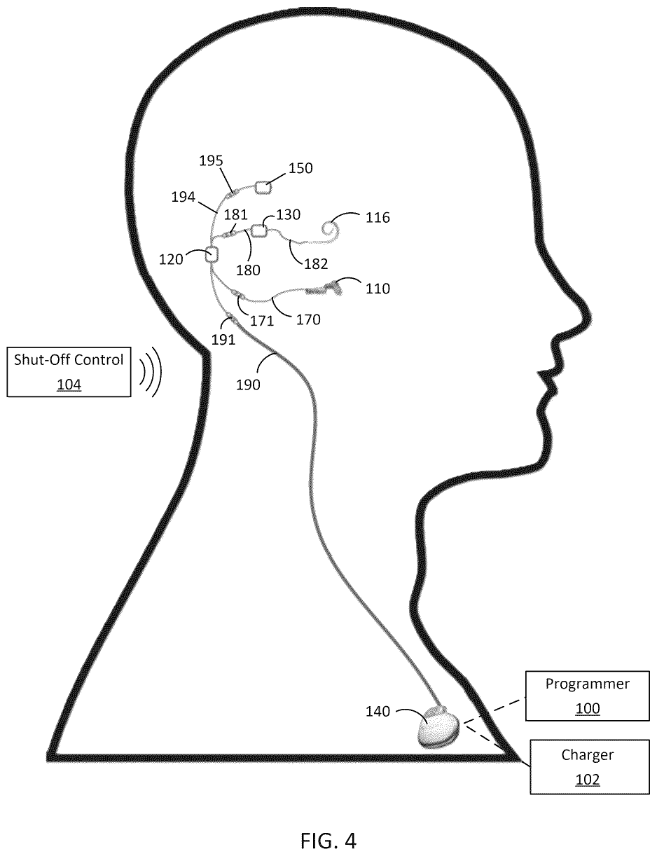

FIG. 4 shows a diagram illustrating an example implantable system including an acoustic stimulator. The acoustic stimulator can be implanted proximate the patient's ossicular chain and can be in communication with a signal processor via lead 194 and detachable connector 195. The signal processor can behave as described elsewhere herein and can be configured to cause acoustic stimulation of the ossicular chain via the acoustic stimulator in in response to input signals from the middle ear sensor according to a transfer function of the signal processor.

The acoustic stimulator of FIG. 4 can be used similarly to the electrical stimulator as described elsewhere herein. For instance, an acoustic stimulator can be mechanically coupled to a patient's ossicular chain upon implanting the system and coupled to the signal processor via lead 194 and detachable connector 195. Similarly to systems described elsewhere herein with respect to the electrical stimulator, if the signal processor requires replacement or repair, the signal processor can be disconnected from the acoustic stimulator (via detachable connector 195) so that the signal processor can be removed without disturbing the acoustic stimulator.

In general, systems incorporating an acoustic stimulator such as shown in FIG. 4 can operate in the same way as systems described elsewhere herein employing an electrical stimulator and cochlear electrode only substituting electrical stimulation for acoustic stimulation.

Some systems can include a hybrid system comprising both an electrical stimulator and an acoustic stimulator in communication with the signal processor. In some such examples, the signal processor can be configured to stimulate electrically and/or acoustically according to the transfer function of the signal processor. In some examples, the type of stimulation used can depend on the input signal received by the signal processor. For instance, in an example embodiment, the frequency content of the input signal to the signal processor can dictate the type of stimulation. In some cases, frequencies below a threshold frequency could be represented using one of electrical and acoustic stimulation while frequencies above the threshold frequency could be represented using the other of electrical and acoustic stimulation. Such a threshold frequency could be adjustable based on the hearing profile of the patient. Using a limited range of frequencies can reduce the number of frequency domains, and thus the number of contact electrodes, on the cochlear electrode. In other examples, rather than a single threshold frequency defining which frequencies are stimulated electrically and acoustically, various frequencies can be stimulated both electrically and acoustically. In some such examples, the relative amount of electrical and acoustic stimulation can be frequency-dependent. As described elsewhere herein, the signal processor transfer function can be updated to meet the needs of the patient, including the electrical and acoustic stimulation profiles.

Additionally or alternatively, while many examples show a middle ear sensor being in communication with an implanted signal processor, in various embodiments, one or more additional or alternative input sources can be included. For instance, in some embodiments, a microphone can be implanted under a user's skin and can be placed in communication with the signal processor (e.g., via a detachable connector such as 171). The signal processor can receive input signals from the implanted microphone and provide signals to the stimulator based on the received input signal and the signal processor transfer function. Additionally or alternatively, systems can include a middle ear sensor as an input source, wherein the middle ear sensor is configured to detect stimuli (e.g., pressure signals) from the wearer's inner ear (e.g., within the cochlear tissue).

With further reference to FIGS. 1 and 4, in some examples, a system can include a shut-off controller 104, which can be configured to wirelessly stop an electrical stimulator 130 from stimulating the patient's cochlear tissue and/or an acoustic stimulator 150 from stimulating the patient's ossicular chain. For example, if the system is malfunctioning or an uncomfortably loud input sound causes an undesirable level of stimulation, the user may use the shut-off controller 104 to cease stimulation from the stimulator 130. The shut-off controller 104 can be embodied in a variety of ways. For example, in some embodiments, the shut-off controller 104 can be integrated into other external components, such as the programmer 100. In some such examples, the programmer 100 includes a user interface by which a user can select an emergency shut-off feature to cease stimulation. Additionally or alternatively, the shut-off controller 104 can be embodied as a separate component. This can be useful in situations in which the patient may not have immediate access to the programmer 100. For example, the shut-off controller 104 can be implemented as a wearable component that the patient can wear at all or most times, such as a ring, bracelet, necklace, or the like.

The shut-off controller 104 can communicate with the system in order to stop stimulation in a variety of ways. In some examples, the shut-off controller 104 comprises a magnet that is detectable by a sensor (e.g., a Hall-Effect sensor) implanted in the patient, such as in the processor and/or the implantable battery and/or communication module 140. In some such embodiments, when the magnet is brought sufficiently close to the sensor, the system can stop stimulation of the cochlear tissue or ossicular chain.

After the shut-off controller 104 is used to disable stimulation, stimulation can be re-enabled in one or more of a variety of ways. For example, in some embodiments, stimulation is re-enabled after a predetermined amount of time after it had been disabled. In other examples, the shut-off controller 104 can be used to re-enable stimulation. In some such examples, the patient brings the shut-off controller 104 within a first distance of a sensor (e.g., a magnetic sensor) to disable stimulation, and then removes the shut-off controller 104. Subsequently, once the patient brings the shut-off controller 104 within a second distance of the sensor, stimulation can be re-enabled. In various embodiments, the first distance can be less than the second distance, equal to the second distance, or greater than the second distance. In still further embodiments, another device such as a separate turn-on controller (not shown) or the programmer 100 can be used to re-enable stimulation. Any combination of such re-enabling of stimulation can be used, such as alternatively using either the programmer 100 or the shut-off controller 104 to enable stimulation or combining a minimum “off” time before any other methods can be used to re-enable stimulation.

In some embodiments, rather than entirely disable stimulation, other actions can be taken, such as reducing the magnitude of stimulation. For example, in some embodiments, the shut-off sensor can be used to reduce the signal output by a predetermined amount (e.g., absolute amount, percentage, etc.). In other examples, the shut-off sensor can affect the transfer function of the signal processor to reduce the magnitude of stimulation in a customized way, such as according to frequency or other parameter of an input signal (e.g., from the middle ear sensor).

In some examples, implantable battery and/or communication module can be used to provide power and/or data (e.g., processing instructions) to other system components via lead 190. Different challenges exist for communicating electrical signals through a patient's body. For example, safety standards can limit the amount of current that can safely flow through a patient's body (particularly DC current). Additionally, the patient's body can act as an undesired signal path from component to component (e.g., via contact with the housing or “can” of each component).

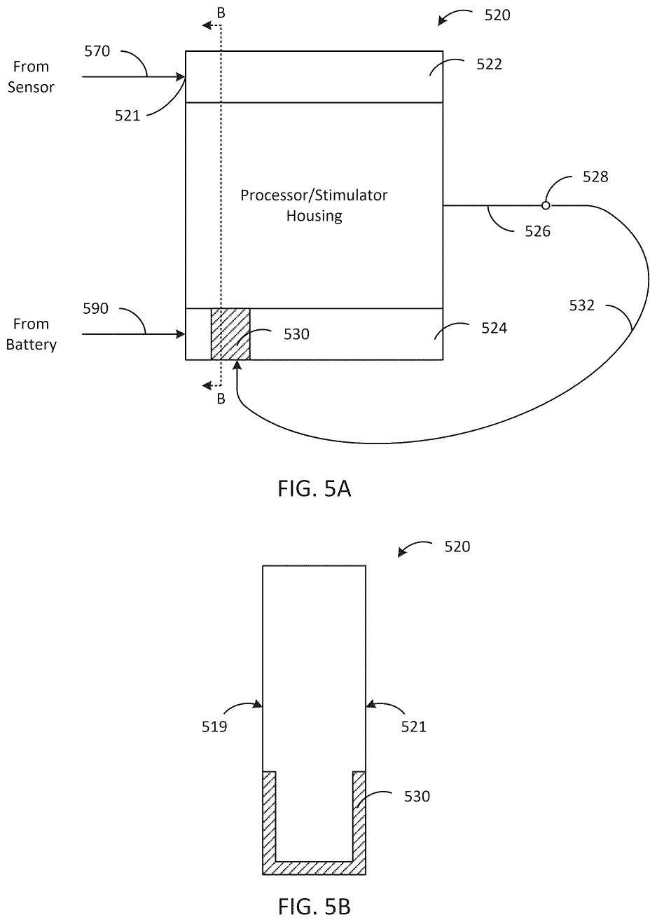

While shown in several embodiments (e.g., FIGS. 1 and 4) as being separate components connected by a lead (e.g., lead 180), in some examples, the processor (e.g., 120) and the stimulator (e.g., 130) can be integrated into a single component, for example, within a hermetically sealed housing. FIG. 5A shows an example illustration of processor and stimulator combined into a single housing. In the example of FIG. 5A, the processor/stimulator 520 receives signal inputs from the sensor (e.g., a middle ear sensor) via lead 570 and power from a battery (e.g., the implantable battery and/or communication module) via lead 590. The processor/stimulator 520 can include headers 522, 524 for receiving leads 570, 590, respectively. In some examples, processor/stimulator 520 comprises a signal processor input 521 configured to interface with lead 570, for example, via a feedthrough.

The processor/stimulator 520 can be configured to receive an input signal from the sensor, process the received input signal according to a transfer function, and output a stimulation signal via electrode 526. Electrode 526 can include one or more contact electrodes (e.g., 528) in contact with a wearer's cochlear tissue to provide electrical stimulation thereto, for example, as described with respect to FIG. 5B.

The processor/stimulator 520 of FIG. 5 includes a return electrode 530 for providing a return path (e.g., 532) for electrical stimulation emitted from electrode 526. The return electrode 530 can be electrically coupled to a ground portion of circuitry within the processor/stimulator 520 to complete a circuit comprising circuitry within the processor/stimulator 520, the electrode 526, the wearer's cochlear tissue, and ground. In some examples, the return electrode 530 comprises an electrically conductive material in electrical communication with circuitry inside the processor/stimulator 520, while the rest of the housing of the processor/stimulator 520 is generally not electrically coupled to internal circuitry.

In some embodiments, the return electrode 530 and the housing of the processor/stimulator 520 comprise electrically conductive materials. For instance, in some examples, the housing comprises titanium while the return electrode 530 comprises platinum or a platinum alloy. Header 524 can generally include a non-conductive biocompatible material, such as a biocompatible polymer. The non-conductive header 524 can provide isolation between the return electrode 530 and the conductive housing of the processor/stimulator 520.

While shown in FIG. 5A as being positioned in the power header 524 of the processor/stimulator 520, in general, the return electrode 530 can be positioned anywhere on the exterior surface of the processor/stimulator 520. In some examples, one or more redundant return electrodes can be included, for example, at or near the interface of the housing and the electrode 526. In some examples, a return electrode can be positioned on a proximal end of the electrode 526 itself. In some embodiments having a plurality of return electrodes (e.g., return electrode 530 and a return electrode on the proximal end of electrode 526), a switch can be used to select which return electrode is used. Additionally or alternatively, a plurality of return electrodes can be used simultaneously.

FIG. 5B shows a simplified cross-sectional view of the processor/stimulator shown in FIG. 5A taken along lines B-B. As shown in FIG. 5B, processor/stimulator 520 includes a housing having a first side 519 and a second side 521 and a return electrode 530 embedded in the housing. Return electrode 530 can comprise a conductive material suitable for contact with a wearer's tissue, such as platinum. In the illustrated example, the return electrode 530 wraps around to both sides of the housing of the processor/stimulator 520 so that the return electrode 530 is coupled to the outer surface of the housing on the first side 519 and the second side 521.

This can facilitate implanting onto either side of a wearer's anatomy, since in some cases, only one side of the processor/stimulator electrically contacts conductive tissue of the wearer while the other side contacts, for instance, the skull of the wearer, and does not easily provide the return path (e.g., 532). Thus, a single processor/stimulator design can be implanted in either side of a wearer's anatomy while providing an adequate return path via a return electrode 530.

In various examples, the return electrode 530 can extend around a perimeter edge of the processor/stimulator 520, as shown in FIG. 5B. In other examples, the return electrode 530 can include sections on either side of the housing and can be connected to one another internally within the housing rather than via a wrap-around contact. Additionally, while shown as being embedded in the housing of the processor/stimulator 520, in some examples, return electrode 530 can protrude outwardly from the housing. Return electrode 530 can generally be any of a variety of shapes and sizes while including an electrical contact section on opposing sides of the housing to provide usability on either side of a wearer's anatomy. In other embodiments, return electrode can be positioned only one side of the housing for a customized right-side or left-side implementation. Some features of a combined processor/stimulator and other cochlear implant system operation are described in U.S. Pat. No. 11,260,220, which is incorporated by reference.

In various examples, a signal processor (e.g., a stand-alone signal processor or a signal processor as part of a combined signal processor/stimulator) can be in communication with an implantable battery and/or communication module, such as via lead 190 as shown in FIG. 1 or lead 590 as shown in FIG. 5A. In various embodiments, communication between the implantable battery and/or communication module and signal processor can be implemented such as described in U.S. Pat. No. 11,260,220, which is incorporated by reference in its entirety. Additionally or alternatively, in various embodiments, communication between other system components (e.g., between separate signal processor and stimulator components) can be carried out in similar ways.

As discussed elsewhere herein, in some embodiments, a cochlear electrode comprises a plurality of contact electrodes that, when the electrode is implanted, contact a corresponding plurality of locations within the cochlea. In an example embodiment, a cochlear electrode comprises 16 contact electrodes, though other numbers are possible.

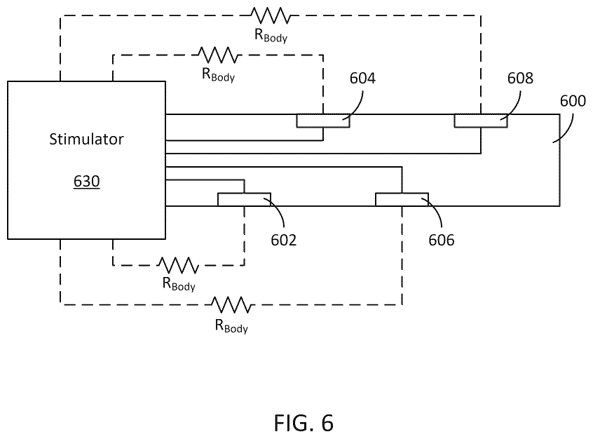

FIG. 6 shows an example cochlear electrode comprising a plurality of contact electrodes. In the illustrated example, cochlear electrode 600 extends from a stimulator 630 and includes contact electrodes 602, 604, 606, 608. As described elsewhere herein, the stimulator can provide electrical signals to one or more such contact electrodes in response to an output from the signal processor according to the transfer function thereof and a received input signal. In some examples, signal processor and stimulator can be separate components coupled by a lead (e.g., lead 180 in FIG. 1). In other examples, signal processor and stimulator can be integrated into a single housing, such as shown in FIGS. 5A and 5B.

Because each contact electrode 602, 604, 606, 608 is in contact with cochlear tissue, each is separated from a return electrode (e.g., 530 in FIGS. 5A and 5B) via the impedance of the patient's tissue, shown as R_{Body}. The term R_{Body} is used to generally represent the resistance and/or impedance of the patient's tissue between various components and does not refer to a specific value. Moreover, each depiction or R_{Body} in the figures does not necessarily represent the same value of resistance and/or impedance as the others.

In some examples, stimulator 630 includes a plurality of source elements that can be configured to source current to and/or sink current from the surrounding tissue via respective contact electrodes. For example, in some embodiments, the stimulator 630 includes as many source elements as the cochlear electrode 600 includes contact electrodes 602, 604, 606, 608. In other examples, the stimulator 630 includes more or fewer source elements compared to the number of contact electrodes. For instance, in some examples, the stimulator 630 includes a single source element configured to source current to and/or sink current via a plurality of contact electrodes, for example, by being in selective communication with a plurality of contact electrodes by way of one or more switches (e.g., one or more mechanical switches or electrical switches). In some such examples, a single source element is configured to sink current from and/or source current to each of the plurality of contact electrodes.

In some examples, during electrical stimulation via the cochlear electrode, a prescribed amount of charge and/or current is sourced to and/or sunk from the tissue via each of one or more contact electrodes. Source elements can be calibrated, for example, as described in U.S. Pat. No. 11,471,689, entitled COCHLEAR IMPLANT STIMULATION CALIBRATION, which is incorporated herein by reference in its entirety. In some examples, the source element is powered via a compliance voltage provided thereto that supports the operation of the source element such that the source element is capable of outputting a prescribed current. Compliance voltages can be determined for individual stimulation paths such as described in U.S. Pat. No. 11,865,339, entitled COCHLEAR IMPLANT SYSTEM WITH ELECTRODE IMPEDANCE DIAGNOSTICS, which is incorporated herein by reference in its entirety.

In some examples, electrical stimulation via the stimulator 630 is accomplished via a stimulation pattern, for example, wherein prescribed stimulation (e.g., current and/or amount of charge) is provided sequentially to each of a plurality of contact electrodes via one or more source elements. As described, such sequential stimulation can be achieved by sourcing and/or sinking current using a plurality of source elements, a single source element and one or more switches, or multiple source elements plus one or more switches.

In some examples, electrical stimulation is provided via each contact electrode sequentially (although not necessarily in a spatially linear sequence), and then the sequence begins again at the first contact electrode of the sequence (though not necessarily providing the same stimulation as in the previous sequence). In some such examples, a stimulation frame is the period of time it takes to deliver stimulation via all of the contact electrodes and a stimulation rate is the rate associated with the stimulation frame (1/frame). For example, if sequential stimulation of all electrodes is completed in a stimulation frame of 100 ms, the stimulation frame rate is 10 Hz. In some embodiments, a stimulation frame is between 1 ms and 2 ms, and the stimulation frame rate is accordingly between 500 Hz and 1000 Hz. In some examples embodiments, the stimulation frame is approximately 1.28 ms, and the stimulation frame rate is approximately 781 Hz.

When stimulation calls for a prescribed current at a given contact electrode, impedance of the tissue being stimulated affects the voltage necessary at the given contact electrode. In some examples, impedances associated with stimulation via each contact electrode can be determined. Such impedances, in combination with a known prescribed current for each contact electrode, can be used to determine the voltage associated with each contact electrode for a given stimulation sequence. The voltage associated with a given stimulation sequence can be considered the voltage pattern for that stimulation sequence. In other examples, a source element works to source or sink a desired current without knowing the voltage needed at the contact electrode to achieve the desired current, but the implant system (e.g., the signal processor) can be configured to determine the voltage at the contact electrode as a desired current is being output. In some such examples, the voltage pattern associated with a stimulation pattern is determined after the stimulation is provided.

In an example implementation, an input source (e.g., a middle ear sensor) detects an acoustic stimulus (e.g., using the anatomy of a wearer) and provides an input signal to the signal processor (e.g., at a signal processor input). The signal processor, programmed with a transfer function, determines a stimulation signal based on the input source and the transfer function, and provides the stimulation signal to the stimulator. In some embodiments, the stimulation signal comprises instructions to cause the stimulator to provide a prescribed stimulation current via one or more of a plurality of contact electrodes. One or more source elements work to source or sink current via the contact electrodes, and the system is configured to determine the voltage at which each prescribed current is output. For instance, in some examples, the signal processor is configured to determine the voltage at the contact electrode as the prescribed current is sourced or sunk.

The voltage associated with the current output to each contact electrode can influence interference within the system. For example, such interference can affect one or more signals throughout the system. This interference can affect how the system receives and processes certain sounds—and how, in turn, a wearer perceives certain sounds—and may require correction or mitigation via various components and/or processes, as discussed in further detail below.

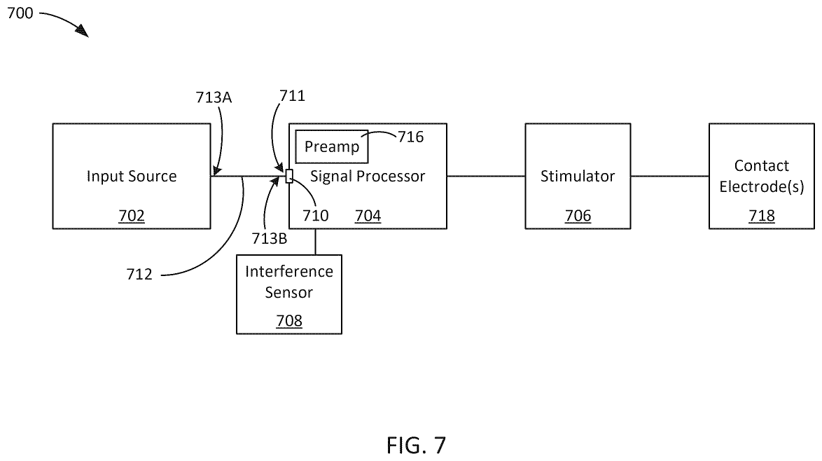

Such interference can impact the system at various components, including, for example, the signal processor. As discussed in further detail above with respect to FIG. 1, the signal processor 120 can be configured to receive an input signal from the input source (for example, middle ear sensor 110). FIG. 7 shows an example diagram illustrating various system components including a signal processor. As shown in the example of FIG. 7, the signal processor 704 can be configured to receive an input signal from an input source 702 (e.g., a middle ear sensor) via a lead 712 and a feedthrough 710 (discussed in further detail below). The signal processor 704 can include a pre-amplifier 716. In some examples, interference related to stimulation can affect the signal processed by the signal processor 704, for example, by being picked up at feedthrough(s) 710 and/or pre-amplifier 716.

In some examples, interference within the cochlear implant system 700 can be associated with the stimulation signal, for example, being associated with a stimulation pattern prescribed by a stimulation signal or a resulting voltage pattern for providing the prescribed stimulation. Some aspects of interference are described in U.S. pre-grant patent publication 2022/0280796, entitled COCHLEAR IMPLANT SYSTEM WITH IMPROVED INPUT SIGNAL-TO-NOISE RATIO and which is incorporated by reference.

As discussed in further detail above, a generated stimulation signal can include a prescribed current being output from the stimulator 706 to at least one of a plurality of contact electrodes 718. The stimulation signal can result in a stimulation pattern applied at the contact electrodes. The stimulation pattern can have an electrical output corresponding to each of the contact electrodes. Interference can be associated with the stimulation pattern resulting from a stimulation signal. For example, interference can correlate to a voltage associated with the system, such as the voltage associated with a stimulation pattern (e.g., a voltage pattern describing voltages at the contact electrodes 718 for providing a prescribed current). Thus, variations in the stimulation pattern can result in variations in interference.

However, the source of interference is not limited to a current being provided from the contact electrodes during electrical stimulation. For example, an open circuit voltage present at one or more contact electrodes alone can cause interference at the signal processor without current being provided from the contact electrodes (e.g., on a benchtop test with no current path from the contact electrodes).

During operation of an implanted system, one or more source elements within the system can operate to output a constant prescribed current during electrical stimulation. An amount of voltage needed to provide the prescribed current can be a function of the impedance of the stimulation path. In some embodiments, a system (e.g., via the signal processor) can be configured to measure or otherwise determine (e.g., via calculation using a known current and impedance) a contact electrode voltage associated with electrical stimulation (e.g., a voltage pattern). One or more source elements may reach a compliance voltage at one or more contact electrodes if impedance of the current flow path associated with stimulation is sufficiently high.

In some cases, higher voltage at the contact electrode(s) leads to higher levels of interference. For example, when the stimulation path impedance and voltage are high, interference in the system may be greater compared to lower voltages required to provide prescribed current through lower impedances.

Interference can also be associated with the stimulation frame rate. For example, in some cases, a fundamental frequency of the interference can be related to the stimulation frame rate. In some embodiments, the fundamental frequency can be equal to the stimulation frame rate. Interference can include the harmonics of the stimulation frame rate.

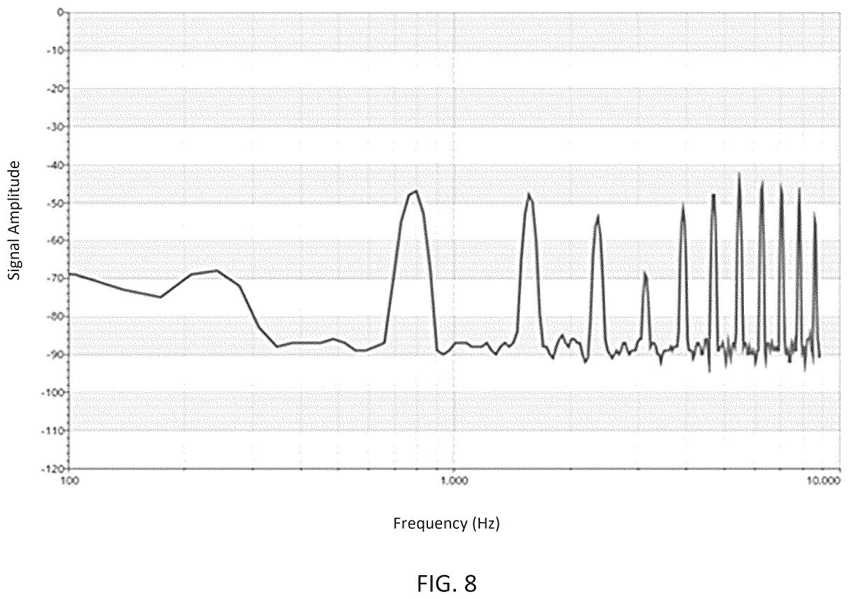

FIG. 8 shows a transform (e.g., FFT) of an input signal showing frequency content of an input signal that includes interference. In the example of FIG. 8, a small amount of signal around 250 Hz represents sound waves picked up by the sensor (e.g., by a middle ear sensor detecting vibrations of a wearer's anatomy caused by incoming sound waves), and higher frequency content (e.g., above 500 Hz in the illustrated example) generally represents interference present in the input signal.

As shown, interference has frequency distribution comprising a fundamental frequency and harmonics of that frequency. In some examples, stimulation parameters, such as voltage, current, stimulation frame rate, etc., can be known (e.g., measured, predetermined, etc.) and can correlate to the frequency content of the interference. For instance, in some embodiments, one or more stimulation parameters can determine the fundamental peak frequency of resulting interference. In the example of FIG. 8, the interference has a fundamental peak at around 781 Hz, and peaks having higher frequency are harmonics of the fundamental frequency.

The magnitude of the peaks in the interference can depend on multiple factors. In some cases, the magnitude of the interference changes as a function of the voltage pattern associated with stimulation (e.g., voltage magnitude required to source or sink a prescribed current). In some cases, the voltage pattern associated with stimulation can affect the relative magnitudes of the frequency components of the interference and/or a noise floor independent of the fundamental and harmonic frequencies of the interference.

Additionally or alternatively, in some cases, the frequency content of the interference changes as a function of the stimulation frame rate (e.g., the fundamental frequency of the interference is equal to the stimulation frame rate frequency and changes as or if the stimulation frame rate changes). In some cases, the stimulation frame rate is fixed at a constant stimulation frame rate until adjusted (e.g., by a clinician).

Information regarding interference (including, for example, information described above) can be determined using a variety of features associated with the system.

As described above, voltage associated with the stimulation signal (e.g., the voltage present at one or more contact electrodes for providing prescribed current according to the stimulation signal) can be used to analyze or predict interference within the system. FIG. 7 shows example components that may be used to measure interference. In some embodiments, the signal processor 704 can be configured to determine a voltage associated with the generated stimulation signal. In some examples, the voltage associated with a stimulation signal (e.g., a voltage pattern required to provide prescribed current) be used to estimate an amount of interference (e.g., a magnitude). Additionally or alternatively, a stimulation frame rate frequency can be used to estimate frequency content of interference. Estimations of magnitudes and/or frequency content of interference based on, for example, a voltage pattern at one or more contact electrodes and/or a stimulation frame rate can be based on an equation or lookup table stored in memory. In some examples, various measurements can be performed to associate various interference information with different stimulation parameters associated with the stimulation signal.

In some examples, a cochlear implant system can include an interference sensor 708 that can be used to measure interference within the system, for example, proximate the signal processor input 711. FIG. 7 shows an interference sensor 708 that can be used to determine interference within a cochlear implant system 700. The interference sensor 708 can be positioned proximate the signal processor 704. The interference sensor 708 can be in communication with the signal processor 704 such that the signal processor can be configured to receive a signal from the interference sensor 708 indicative of the interference associated with stimulation. In various examples, the interference sensor can be separate from or integral with the signal processor housing. In some examples, interference sensor comprises an electrode and/or a conductor that acts as an antenna to pick up electrical interference associated with stimulation.

The interference sensor 708 can interact with various components and/or elements of the system 700. The interference sensor 708 can be configured to sense interference at various locations associated with the signal processor 704, for example, including the signal processor input 711. The signal processor 704 can be configured to determine an amount of interference at the signal processor input 711 associated with a stimulation signal using the interference sensor. For example, the interference sensor 708 can be configured to output an interference signal based on electrical signals received at the interference sensor 708. The signal processor 704 can be configured to receive the interference signal from the interference sensor 708 and determine the amount of interference at the signal processor input 711 based on the interference signal received from the interference sensor 708. The signal processor can be configured to associate the measured interference with a stimulation signal being output while the interference signal is received, such as associating the measured interference with one or more contact electrode voltages.

The signal processor can be configured to determine information regarding interference in other ways. For instance, in some embodiments, the input signal at the signal processor input 711 can indicate interference within a cochlear implant system 700. For example, the signal processor can analyze or evaluate the input signal while undergoing various processes to identify interference within the system 700. The signal processor 704 can be configured to measure a signal at the signal processor 704 input when the stimulator 706 is not providing any output to the cochlear electrode 718. The signal processor 704 can cause the stimulator 706 to output electrical stimulation to the cochlear electrode 718 and measure a signal at the signal processor input 711 when the stimulator 706 is outputting the electrical stimulation to the cochlear electrode 718. Electrical stimulation can be a function of an input signal received at the input source or an independent electrical stimulation (e.g., electrical stimulation using a predetermined voltage or current level). In some examples, the signal processor input measurements when electrical stimulation is and is not provided are both performed when a constant acoustic stimulus is received at the input source (e.g., a pure tone or other predetermined constant sound provided, for example, by a fitting hub or other external device with a speaker, or in an intentionally silent environment).

Determining the amount of interference at the signal processor input associated with the stimulation signal can include comparing the signal at the signal processor input when the stimulator is outputting the electrical stimulation to the cochlear electrode to the signal at the signal processor input when the stimulator is not outputting electrical stimulation to the cochlear electrode. Comparison of the two signals can be indicative of interference within the system. Information identified in comparison of the two signals can be saved in a lookup table, for example, associated with the stimulation signal causing any detected interference. Determining an amount of interference associated with the stimulation signal can include referencing such an entry in a lookup table.

In some examples, determining an amount of interference comprises comparing a difference in input signals at the signal processor when a stimulus is provided to the input source versus when minimal or no stimulus is provided to the input source. For instance, in some cases, in a quiet room, stimulation levels are low, but as noise in the environment increases, the input signal to the signal processor increases and the stimulation level increases. In some cases, once input signal rises above a threshold input level, a resulting voltage pattern for providing a prescribed stimulus rises above a threshold voltage level such that interference is picked up in the input signal, which can cause detectable interference.

The difference in the input signal between before the input signal crossed the threshold input level (e.g., during a time of no or low levels of acoustic input) and after the input signal rises above the threshold voltage can be used to estimate an amount of interference in the input signal. In some cases, even a small amount of acoustic input can cause the input signal to cross the threshold input level, for example, in some examples, tapping on a table in an otherwise quiet room can cause the input signal to rise above a threshold input level and cause interference.

In some cases, interference picked up in the input signal, even at a low level, creates feedback where the picked up interference causes corresponding contributions to stimulation, which is in turn picked up in the input signal and leads to additional stimulation. In some cases, this also leads to continued interference and stimulation even after the initial acoustic input that initiated the stimulation has subsided or changed.

Additional information may be used in determining information regarding interference at the signal processor input. For example, determining the amount of interference at the signal processor input associated with the stimulation signal can include determining a fundamental frequency of a stimulation artifact based on the stimulation signal, for instance, based on the stimulation frame rate.

Various approaches can be used to mitigate interference within the system. For example, the signal processor 704 can be configured to modify the input signal received from the input source 702. Such a modification can be determined based on the amount of interference at the signal processor input 711 (e.g., as measured and/or determined using a lookup table based on a previous stimulation). In some embodiments, modifying the input signal can include subtracting a determined amount of interference from the input signal to create a modified input signal. The signal processor can process the modified input signal according to the transfer function to generate a stimulation signal based on the modified input signal with reduced interference. The signal processor can output the stimulation signal to the stimulator for causing the stimulator to output electrical stimulation via the cochlear electrode.

In addition or as an alternative to subtracting interference from the input signal, in some embodiments, the signal processor 704 can be configured to update the transfer function of the signal processor based on the determined amount of interference at the signal processor input 711 associated with the stimulation signal. The updated transfer function can minimize the effects of the interference received at the signal processor in stimulation signal output from the signal processor (e.g., by reducing a gain associated with frequencies present in the interference).

After determining the amount of interference at the signal processor input 711 associated with the stimulation signal, the signal processor 704 can receive a subsequent input signal from the input source 702 at the signal processor input 711. The signal processor 704 can update its transfer function and/or modify the subsequent input signal to compensate for the determined amount of interference at the signal processor input 711 associated with the stimulation signal. In various examples, the signal processor can (i) modify the subsequent input signal to generate a modified subsequent input signal and apply the transfer function to modified subsequent input signal, (ii) update the transfer function and apply an updated transfer function to the subsequent input signal, or (iii) modify the subsequent input signal to generate a modified subsequent input signal, update the transfer function, and apply an updated transfer function to the modified subsequent input signal.

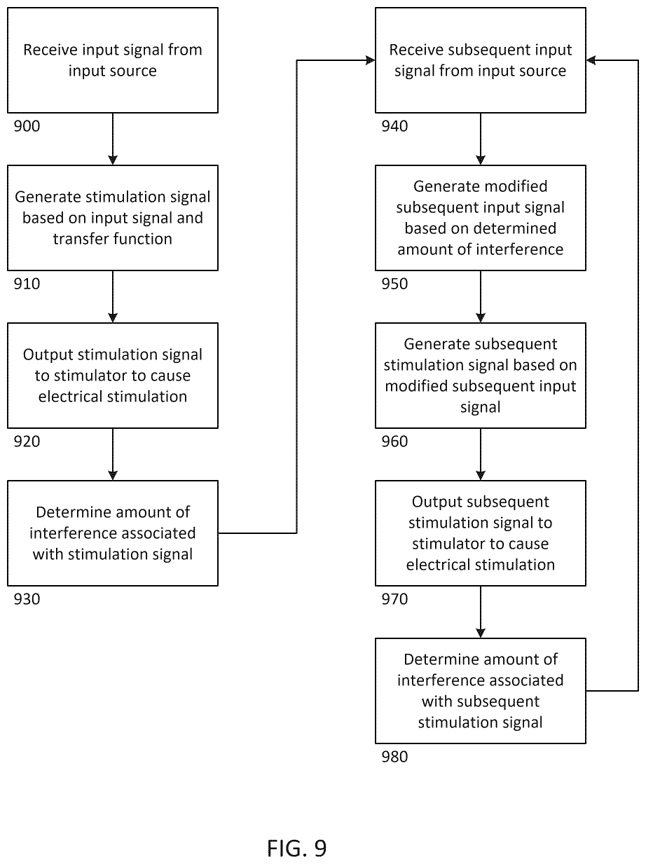

FIG. 9 shows an example process of addressing interference by modifying an input signal. As shown, the process of FIG. 9 includes receiving an input signal from an input source (900), generating a stimulation signal based on the input signal and a transfer function (910) and outputting the stimulation signal to a stimulator to cause electrical stimulation (920). The process further includes determining an amount of interference associated with the stimulation signal (930).

The process includes receiving a subsequent input signal from the input source (940) and generating a modified subsequent input signal based on the determined amount of interference (950), for instance, the amount of interference determined at step 930. In some examples, generating the modified subsequent input signal comprises subtracting the determined interference from the subsequent input signal.

The process includes generating a subsequent stimulation signal based on the modified subsequent input signal and the transfer function (960) and outputting the subsequent stimulation signal the stimulator to cause electrical stimulation (970). The process includes determining amount of interference associated with the subsequent stimulation signal (980), and repeats with receiving a subsequent input signal from the input source. During subsequent iterations of steps 940-980, generating modified subsequent input signals at step 950 can be based on the most recently determined amount of interference associated with a stimulation signal at step 980 of the previous iteration.

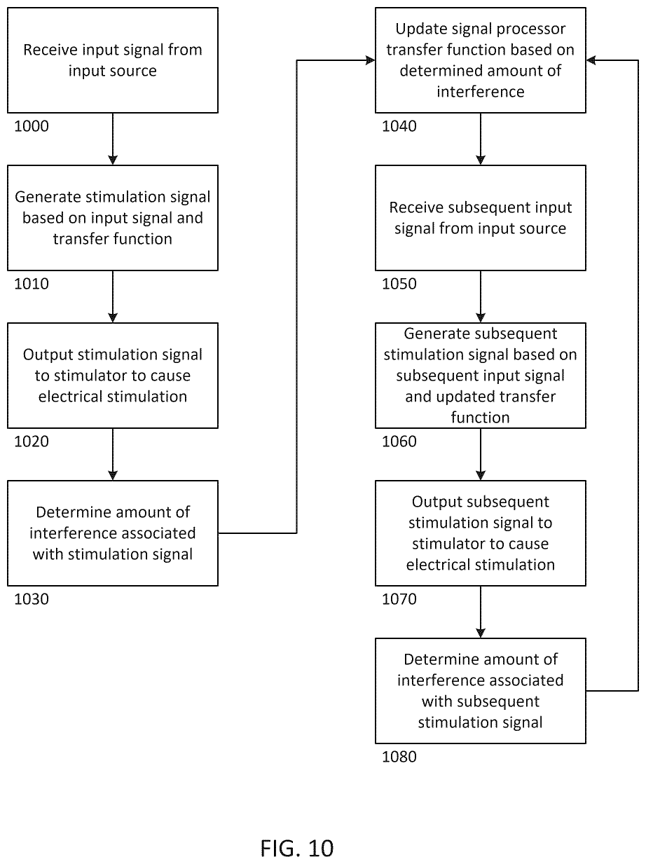

FIG. 10 shows an example process of addressing interference by updating a signal processor transfer function. As shown, the process of FIG. 10 includes receiving an input signal from an input source (1000), generating a stimulation signal based on the input signal and a transfer function (1010) and outputting the stimulation signal to a stimulator to cause electrical stimulation (1020). The process further includes determining an amount of interference associated with the stimulation signal (1030).

The process includes updating the signal processor transfer function based on the determined amount of interference (1040), receiving a subsequent input signal from the input source (1050), and generating a subsequent stimulation signal based on the subsequent input signal and the updated transfer function (1060).

The process includes outputting the subsequent stimulation signal the stimulator to cause electrical stimulation (1070) and determining amount of interference associated with the subsequent stimulation signal (1080), and repeats with receiving a subsequent input signal from the input source. During subsequent iterations of steps 1040-1080, updating the signal processor transfer function at step 1040 can be based on the most recently determined amount of interference associated with a stimulation signal at step 1080 of the previous iteration.

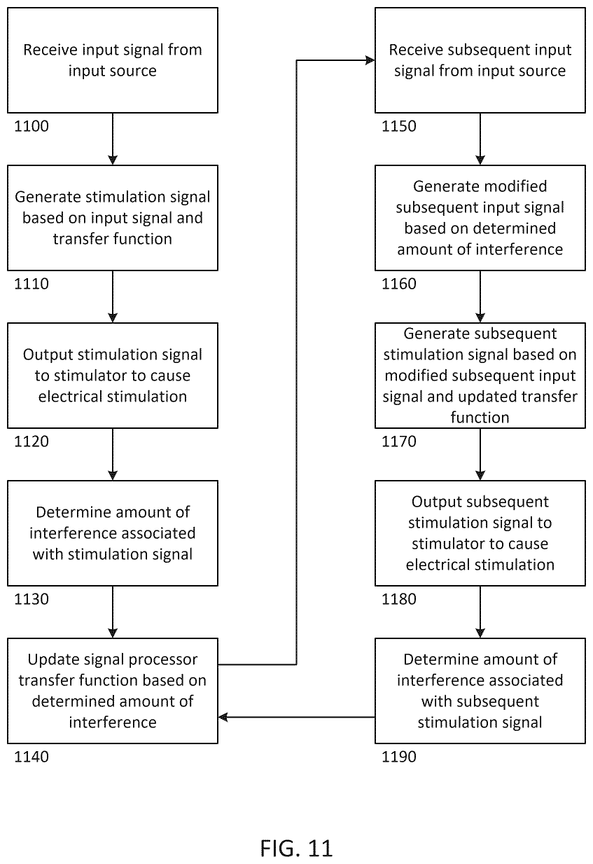

FIG. 11 shows an example process of addressing interference by modifying an input signal and updating a signal processor transfer function. As shown, the process of FIG. 11 includes receiving an input signal from an input source (1100), generating a stimulation signal based on the input signal and a transfer function (1110) and outputting the stimulation signal to a stimulator to cause electrical stimulation (1120). The process further includes determining an amount of interference associated with the stimulation signal (1130).

The process includes updating the signal processor transfer function based on the determined amount of interference (1140), receiving a subsequent input signal from the input source (1150), and generating a modified subsequent input signal based on the determined amount of interference (1160), for instance, the amount of interference determined at step 1130. In some examples, generating the modified subsequent input signal comprises subtracting the determined interference from the subsequent input signal.

The process further includes generating a subsequent stimulation signal based on the subsequent input signal and the updated transfer function (1170), outputting the subsequent stimulation signal the stimulator to cause electrical stimulation (1180), and determining amount of interference associated with the subsequent stimulation signal (1190). The process and repeats with updating a signal processor transfer function based on the determined amount of interference at step 1140, for example, the most-recently determined amount of interference determined at step 1190 and receiving another subsequent input signal from the input source. During subsequent iterations of steps 1140-1190, updating the signal processor transfer function at step 1140 and generating a modified subsequent input signal at step 1160 can be based on the most recently determined amount of interference associated with a stimulation signal at step 1190 of the previous iteration.

In an example embodiment, a signal processor receives an input signal from an input source at time t₁, and is configured to generate a stimulation signal associated with the input source at time t₁ and provide the stimulation signal to the stimulator to cause stimulation via the cochlear electrode. The signal processor can also determine an amount of interference at the signal processor input associated with the stimulation signal based on the input signal received at time t₁, for example, such as described herein.

In embodiments where the signal processor is configured to modify a subsequent input signal, the signal processor can receive a subsequent input signal at time t₂, later than time t₁. The signal processor can be configured to modify the input signal received at time t₂ to compensate for the interference determined based on the stimulation at time t₁. For example, by subtracting the determined interference from the subsequent input signal received at time t₂, to generate a modified subsequent input signal. The signal processor can process the modified subsequent input signal to generate a stimulation signal.

In embodiments where the signal processor is configured to adjust the transfer function based on the determined amount of interference, the signal processor can update the transfer function and receive a subsequent input signal at time t₂, later than time t₁. The signal processor can be configured to process the subsequent input signal via the updated transfer function to generate a stimulation signal.

In some cases, interference correction for a received input signal at a given time (e.g., t₂) is based on determined interference associated with a stimulation signal from a previous time (e.g., t₁). However, the timescale of interference associated with a given is short compared to typically changing acoustic environments. For instance, in an example embodiment, a stimulation frame rate is approximately 781 Hz, so the time it takes to deliver stimulation to al electrodes within the stimulation frame is approximately 1.28 ms. In some embodiments, interference compensation for a subsequent stimulation frame based on the stimulation in the previous stimulation frame will lag the stimulation used to determine the interference by less than 10 ms, in some embodiments by less than 5 ms, and in some embodiments, between 1 and 2 ms. Even if interference associated with a given is used to compensate for interference associated with subsequent input signals, the timescales involved are small enough such that any lag would not likely be perceived.

In some examples, the signal processor can be configured to determine the amount of interference at the signal processor input over time (e.g., for each stimulation frame or at some other predetermined frequency). The signal processor 704 can apply an adaptive filter to the received input signal based on the determined amount of interference to reduce or eliminate the interference at the signal processor input over time, for example, for subsequent input signals received from the input source.

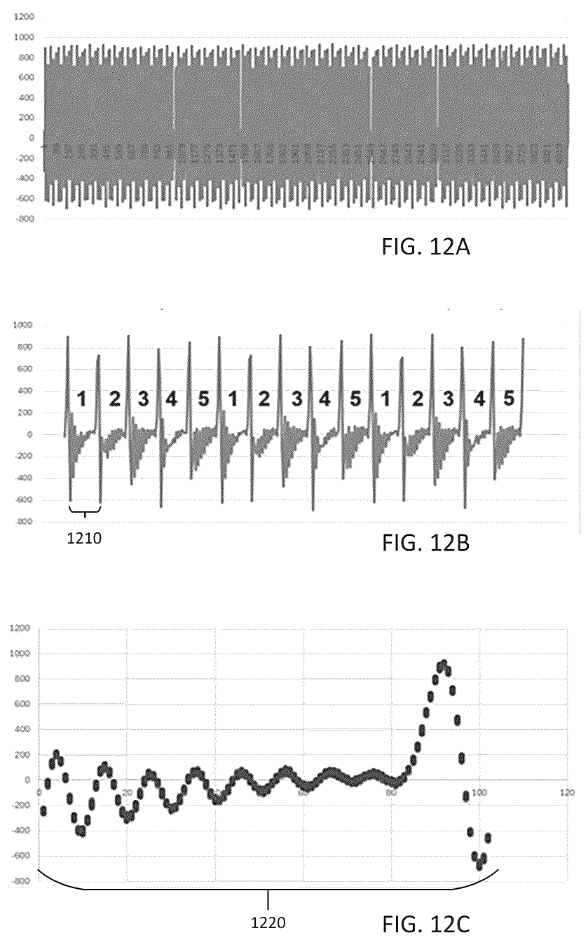

In some embodiments, determining an amount of interference and/or addressing the interference can involve analyzing signals from an input source in the time domain. FIG. 12A shows an example signal from an input source while the system outputs a stimulation pattern at a stimulation frame rate. As shown, a sampled 4096 data points of the input source data is very noisy over a period of approximately 0.3 s. FIG. 12B shows a zoomed-in section of the signal in FIG. 12A, showing 300 data points of the signal over a period of approximately 0.02 s. The length of one stimulation frame 1210 is shown in the example. The signal appears to repeat every 5 stimulation frames. However, this is a byproduct of the sampling rate of the data rather than the noise itself. FIG. 12C shows 4096 data points repeatedly overlaid on a single time period of one stimulation frame 1220, approximately 1.28 ms in the illustrated example. As shown, when overlaid and aligned over time, each stimulation frame has a consistent artifact pattern. Noise attributable to stimulation patterns over a stimulation frame rate appears to repeat consistently over short periods of time. In some cases, such properties of the noise make the noise suitable for being estimated by a moving average filter, and the estimate can be subtracted from the input signal to eliminate the noise.

As shown in FIG. 12C, the noise present in a given stimulation frame approximates a damped sinusoid. The noise patterns tends to change at much longer time scales compared to sound information received at an input source.

In some embodiments, a Kalman filter can be used to fit stimulation noise to a model. For example, a Kalman filter can be programmed to recognize a damped sinusoid pattern in an input signal from the input source. The Kalman filter can use a mathematical model to identify the noise of that pattern within the input signal, and the modeled noise can be removed from the input signal to address the noise.

In other embodiments, an adaptive filter can be used that does not require a particular model. The adaptive filter can be configured to recognize a repeating pattern within a signal, such as a persistent noise signal. If such a signal changes slowly over time, or changes and then again remains constant, the adaptive filter can be configured such that it will recognize and adapt to the changed repeating pattern. In an example implementation, a filter can be configured to sum or average the signal with itself over several periods of repetition (e.g., over several stimulation frames). Persistent noise that repeats over such periods will be consistently present within each set of data, and everything that is not repetitive over such a period will be canceled or otherwise suppressed relative to the repetitive aspect of the signal (the repeating noise). Such an adaptive filter can be used to construct an approximate shape of the repeating noise signal in the time domain, which can be used to remove such noise from the input signal.

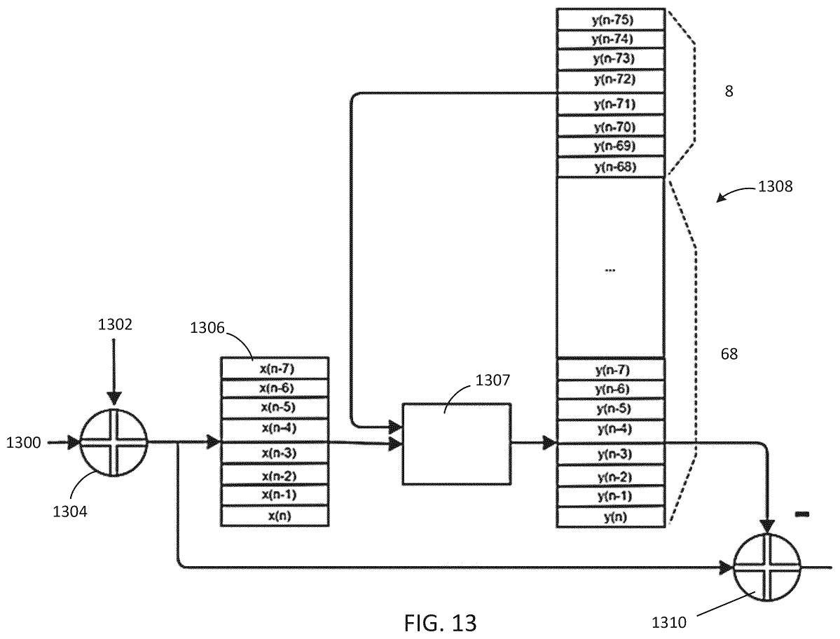

FIG. 13 shows an illustration of an example adaptive filter. As shown, a desired signal 1300, such as a sound signal in an environment of a wearer of a cochlear implant system, and a stimulation artifact 1302 are combined at a sensor 1304, such as a middle ear sensor. The stimulation artifact 1302 can be due to, for example, voltage present at one or more contact electrodes of a cochlear implant system. In the example shown, the stimulation artifact and desired signal combine at the sensor to form a combined signal 1306.

In an example, an adaptive filter uses a number of “bins,” each of which includes a moving average of data. When a new data point is acquired (e.g., in the combined signal 1306), the new data point is used to update a bin of the filter, the value in that bin is subtracted from the new data point to create a corrected data point. When a next data point (e.g., of combined signal 1306) is acquired, that data point is used to update the next bin, whose value is then subtracted from that next data point to create a corrected next data point. Such a process continues through all bins of the filter, and then the filter restarts at a first bin. The filter has an associated filter frequency and corresponding filter period, where the period of the filter is the amount of time it takes to update each of the bins before restarting and re-updating the same bins. In some embodiments, the filter is programmed with a filter frequency corresponding to the period of a stimulation frame such that each bin of the filter is updated once during each stimulation frame.

In the example of FIG. 13, the combined signal 1306 has eight data points, each labeled x(n) . . . x(n−7). The adaptive filter of FIG. 13 has 68 bins of estimated stim artifact data 1308, with each bin being representative of a different point in time within a stimulation frame. Each estimated stimulation artifact data point is labeled in the illustrated figure y(n) . . . y(n−75). Since there are 68 bins, for a given integer A, y(n−A) corresponds to an updated value in the same bin (and occurring at the same relative time within a stimulation frame) as y(n−A−68) after the filter has progressed through all 68 bins. That is, in the example with 68 bins, y(n−A) represents an updated stimulation artifact value of a previous bin value of y(n−A−68). For example, if y(n−2) represents an updated estimated stimulation artifact for the bin whose previous estimated stimulation artifact was y(n−70).

As shown in the illustrated example, a data point from the combined signal 1306 (e.g., a data point x), is modified by subtracting an estimated stim artifact value (e.g., a data point y) at a signal modification step 1310.

In the example of FIG. 13 and as discussed above, the adaptive filter can be updated using the combined signal data acquired from the sensor. A smoothing filter 1307 (e.g., a one-pole smoothing filter) can be implemented for using existing estimated stim artifact values (e.g., y(n−68) through y(n−75)) and combined signal values (e.g., x(n) through x(n−7)) from the sensor to update the estimated stimulation artifact (e.g., y(n) through y(n−7)). Two example techniques for identifying a stimulation artifact (y) are outlined below:

### Example 1

$$y ( n - k ) = α \times y ( n - k - 68 ) + ( 1 - α ) \times n ( n - k )$$

$$where k = 0 , 1 , … , 7.$$

In this example, a stimulation artifact data point y(n−k) is a weighted average of the previous bin value y(n−k−68) and a corresponding combined signal data point x(n−k). After a given stimulation artifact estimated value for a given bin is updated, a next bin is updated in a similar manner. Once all bins have been updated, the process continues over by once again updating a first bin and the process continues.

### Example 2

if x(n−k) > y(n−k−68), then y(n−k) = y(n−k−68) × F
else if x(n−k) ≤ y(n−k−68), then y(n−k) = y(n−k−68) / F
where k = 0, 1, ..., 7 and 1.0001 ≤ F ≤ 1.01

In this example, if a combined signal value x(n−k) is larger than a previous value of estimated stimulation artifact for a given bin y(n−k−68), then updating the estimated stimulation artifact involved increasing the estimated stimulation artifact for that bin (multiplying by F>1). If a combined signal value x(n−k) is smaller than a previous value of estimated stimulation artifact for a given bin y(n−k−68), then updating the estimated stimulation artifact involved decreasing the estimated stimulation artifact for that bin (dividing by F>1).

In the illustrated and described examples with respect to FIG. 13, the combined signal 1306 comprises eight data points, although other values are possible. Eight data points can be useful when a processing device (e.g., an implanted signal processor) is configured to process eight data points at a time.

In the illustrated and described examples with respect to FIG. 13, the adaptive filter has 68 bins, although other values are possible. In an example embodiment, the number of bins corresponds to a number of data points associated with an integer number of stimulation frames. For instance, in an example embodiment, a stimulation frame rate and data collection rate are such that three stimulation frames occur in the amount of time it takes to collect 68 data points such that every 68^{th} data point corresponds to approximately the same time within a stimulation frame even if not necessarily from consecutive stimulation frames.

Suitable numbers of data points can vary based on data collection rates and stimulation frame rates. In some examples, the number of bins of the adaptive filter is the minimum number of bins that correspond to an integer number of stimulation frames.

In both Examples 1 and 2, the adaptive filter will evolve over time, adjusting to eliminate consistently present and repeating artifacts having the frequency of the filter or a harmonic thereof. In some examples, upon initiation, the adaptive filter will take a short amount of time to build up enough of a recognition of the stimulation artifact to maximize the filter's reduction of the artifact. The adaptive filters as shown in the above examples will continue to run and update over time. Since the artifact is likely to be the only repeating aspect of the signal over each of several iterations at the frequency of the filter, the estimated artifact resulting from updating the bins will approach the constant, repeating artifact having a frequency of the filter or a harmonic thereof. Such artifact will be eliminated more and more over time at the signal modification step. In some embodiments, an adaptive filter will take approximately 1 second to accurately estimate the stimulation artifact and substantially reduce or eliminate the artifact from the output via the signal modification.

Such time domain adaptive filters identify signals having frequency of the filter frequency and harmonics thereof, and can be used to eliminate such signals (e.g., stimulation artifact signals over a stimulation frame). The filtering tends to have a very narrow effect in the frequency domain, narrowly recognizing/eliminating signals within a very narrow frequency band around a filter frequency (and harmonics thereof). Thus, the filter has very little effect on desirable audio signals outside of the stimulation artifacts.

Additionally, even in cases where the stimulation artifact has a particular frequency content and the adaptive filter has evolved to remove the stimulation artifact, a desired signal (e.g., a sound detected by an input source) having similar frequency content will typically be unaffected by the adaptive filter. Rather than only attenuating a particular frequency corresponding to the stimulation artifact (e.g., in the frequency domain) the time domain adaptive filter builds an estimation of the stimulation artifact itself and happens to filter in narrow frequency bands. But because the time domain adaptive filter takes a number of iterations to recognize constantly repeating signal (approximately 1 second in some examples), and an audio signal of interest tends to change on a timescale much shorter than that, even portions of audio signals of interest having frequency content in the frequency bands associated with the stimulation artifact will not be significantly attenuated by the time domain adaptive filter.

The time domain adaptive filter can be programmed with a filter frequency. When a stimulation frame rate is known, the time domain adaptive filter can be programmed with a filter frequency corresponding to the stimulation frame rate. Stimulation artifacts having frequency content corresponding to the stimulation frame rate will be recognized via the time domain adaptive filter and removed from incoming combined signals such as described herein. The time domain adaptive filter can recognize and eliminate or reduce contribution of the stimulation artifact while generally leaving an input signal coming from an input source unaffected, even signals having frequency content overlapping the stimulation frame rate. Accordingly, time domain adaptive filters can effectively reduce or eliminate stimulation interference while being unlikely to affect the perception of speech or other sounds of interest to be perceived by a wearer of a cochlear implant system.

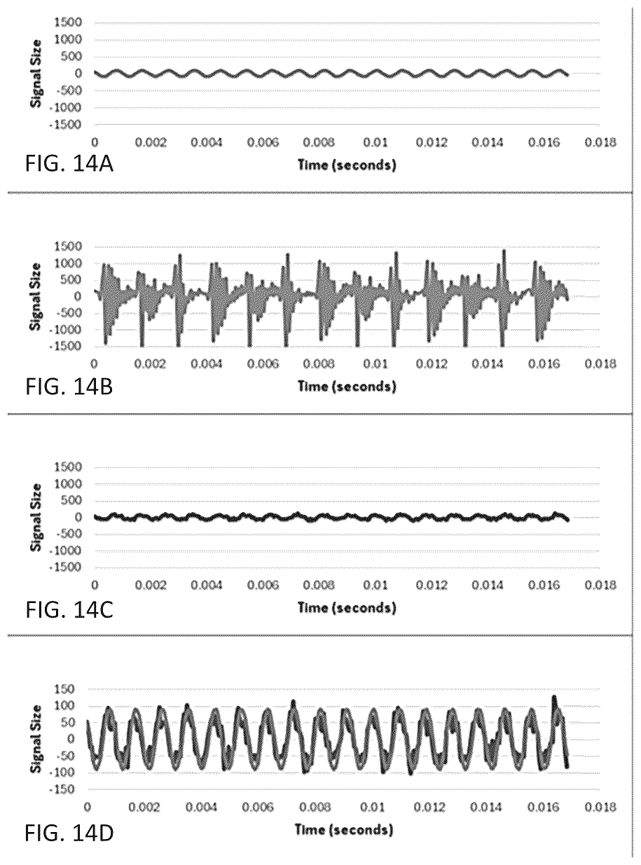

Additionally, signals having a constant frequency that is outside of the filter frequency are generally unaltered by the time domain adaptive filter. FIGS. 14A-D show an example application of a time domain adaptive filter removing a stimulation artifact added to a pure tone sound signal. With reference to the illustration of FIG. 13, FIG. 14A shows a desired signal prior to the addition of a stimulation artifact thereto in the form of a pure 1 kHz sine wave. FIG. 14B shows a combined signal, which includes a stimulation artifact combined into the desired signal of FIG. 14A. After applying a time domain adaptive filter such as discussed above with respect to FIG. 13 with a filter frequency corresponding to a stimulation frame rate, and after the filter has run sufficiently long to build a profile of any recurring signal having a frequency corresponding to the filter frequency, the time domain adaptive filter can be used to remove the stimulation artifact such as discussed above. FIG. 14C shows the signal resulting from applying the time domain adaptive filter to the signal of FIG. 14B, and FIG. 14D shows the initial signal of FIG. 14A overlaid onto the result of the filter shown in FIG. 14C. Despite the large amount of noise added to the signal by the stimulation artifact as shown in FIG. 14B, the result of the time domain adaptive filter restores the signal to approximately match the original signal.

Additionally or alternatively to performing various signal correction techniques described herein, in some embodiments, a stimulation pattern can be implemented to help reduce the impact of stimulation artifacts. In an example embodiment of traditional stimulation, a cochlear electrode includes 16 contact electrodes, and during stimulation, each contact electrode outputs provides electrical stimulation corresponding to a different range of frequencies of the received input. For instance, an input signal received from an input source (e.g., a middle ear sensor) can be divided into a plurality of frequency bands, and electrical stimulation at individual contact electrodes can be provided for a given frequency band. Each contact electrode provides separate stimulation channels having separate frequency content.

However, in some embodiments, multiple contact electrodes can be used to provide electrical stimulation corresponding to the same or overlapping audio frequency bands. In some examples, a received input signal is divided into eight distinct frequency bands, and of 16 contact electrodes pairs of two contact electrodes each deliver electrical stimulation corresponding to one of the eight distinct frequency bands.

If paired channels contribute differently to the overall stimulation artifact, then using both channels to provide stimulation for a given frequency channel provides double the stimulation (e.g., in the form of charge delivered to cochlear tissue) for a given frequency band, but will less than double the artifact for the given frequency band. In general, the noise due to stimulation artifacts will increase by a factor of approximately √2 while the stimulation intensity doubles, effectively improving the signal to noise ratio for each frequency range. Stimulation for a given frequency across multiple contact electrodes can also increase perceived loudness in such a frequency range, for example, due to increased charge delivered via stimulation associated with the given frequency range.

Further additional or alternative approaches for reducing the impact of noise due to stimulation artifacts are possible. Turning back to FIG. 7, various mechanical features can be integrated into the system to reduce interference. For example, the system 700 can include a lead 712 coupled to the signal processor 704 and the input source 702. The lead 712 can have a first end 713A and a second end 713B. The first end 713A of the lead 712 can be coupled to the input source 702. As discussed in further detail with respect to FIG. 1, the input source 702 can be configured to provide the input signal to the signal processor 704 via the lead 712.

The second end 713B of the lead 712 can be operatively connected to the signal processor 704. In some embodiments, the second end 713B of the lead 712 can be removably coupled to a feedthrough 710 of the signal processor 704. The feedthrough 710 can be configured to couple to an end 713 of the lead 712 such that the signal processor 704 receives the input signal from the input source 702 via the lead 712 and the feedthrough 710.

As described, the signal processor 704 can include a signal processor input 711. The signal processor input 711 can include a feedthrough 710 coupled to the second end 713B of the lead 712 such that the signal processor input 711 is in communication with the input source 702 via the lead 712 and feedthrough 710. Feedthrough 711 can be included in a header (e.g., 522 in FIG. 5A) of signal processor 704. Feedthroughs, such as those displayed in FIG. 2, can be incorporated into various components of the system.

Additionally or alternatively, in some examples, the signal processor 704 can include a pre-amplifier 716 in electrical communication with the feedthrough 710. The pre-amplifier 716 can receive the input signal from the input source 702 via the pre-amplifier 716.

Such interference mitigation measures are compatible with variations within the system 700. For example, in some embodiments of the cochlear implant system, the lead can include at least two conductors 720. Each conductor 720 can be operatively connected with other components of the system. For example, the feedthrough 710 can be configured to couple to an end of a first of the at least two conductors 720. The signal processor 704 can include a second feedthrough (e.g., such as that of FIG. 2) configured to couple to a second of the at least two conductors.

In some embodiments, a sensor (e.g., a middle ear sensor or a microphone) has a positive output and a negative output. In some cases, the sensor can be a differential sensor. In some examples, an output (e.g., the negative output) can be grounded such that the sensor operates in a single-ended configuration. The positive output and negative output can be provided to inputs of a differential amplifier which can be used to reject common mode noise and prepare the signal for processing and stimulation signal generation. Interference caused by electrical stimulation or other electrical potentials of the system, such as described herein, can be capacitively coupled to the positive and negative outputs of the sensor, for example, by coupling into a header in a housing that houses the signal processor configured to receive input signals from the sensor. The interference can be considered an aggressor signal, and can behave as a potential outside of the housing with respect to device ground that couples into the header.

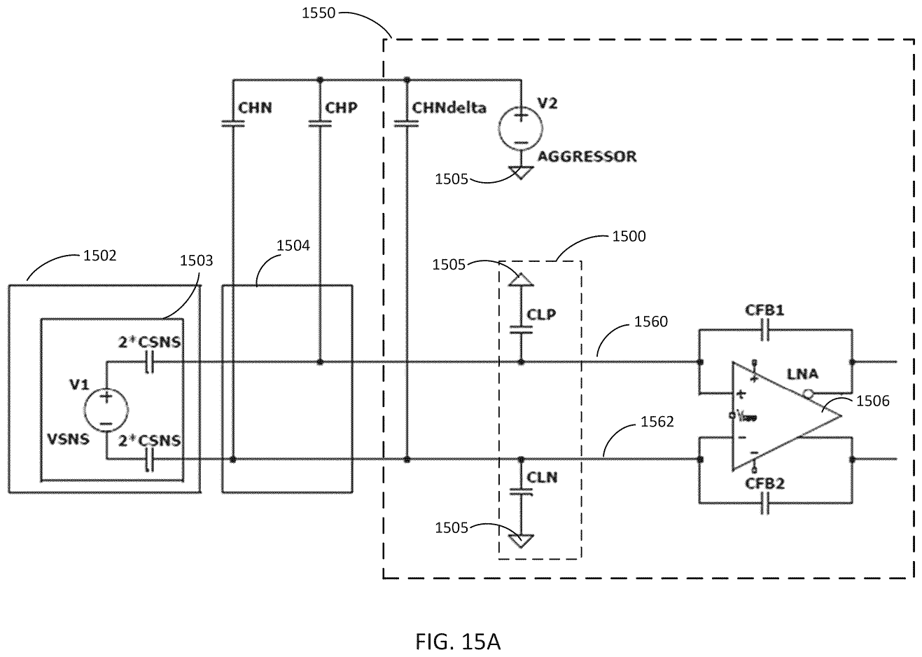

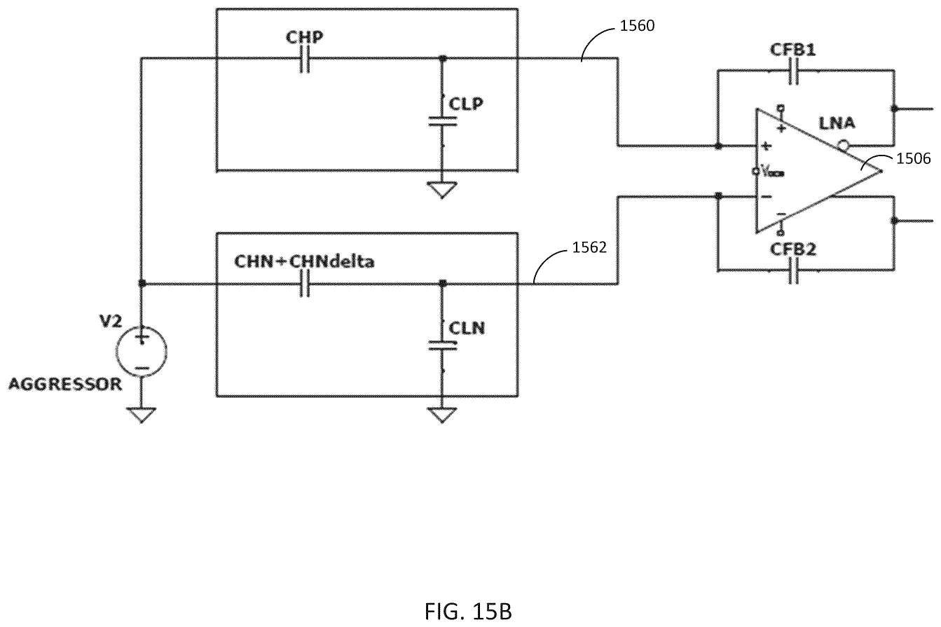

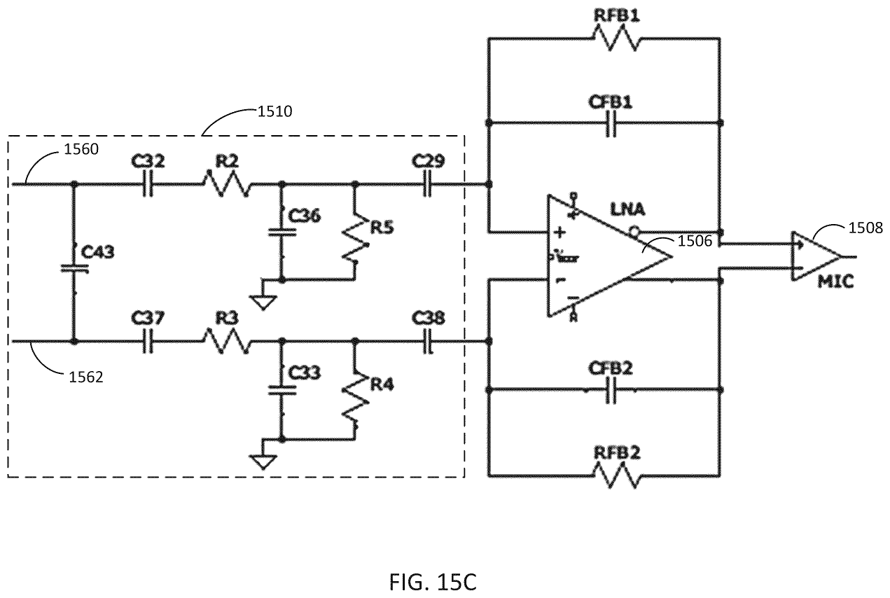

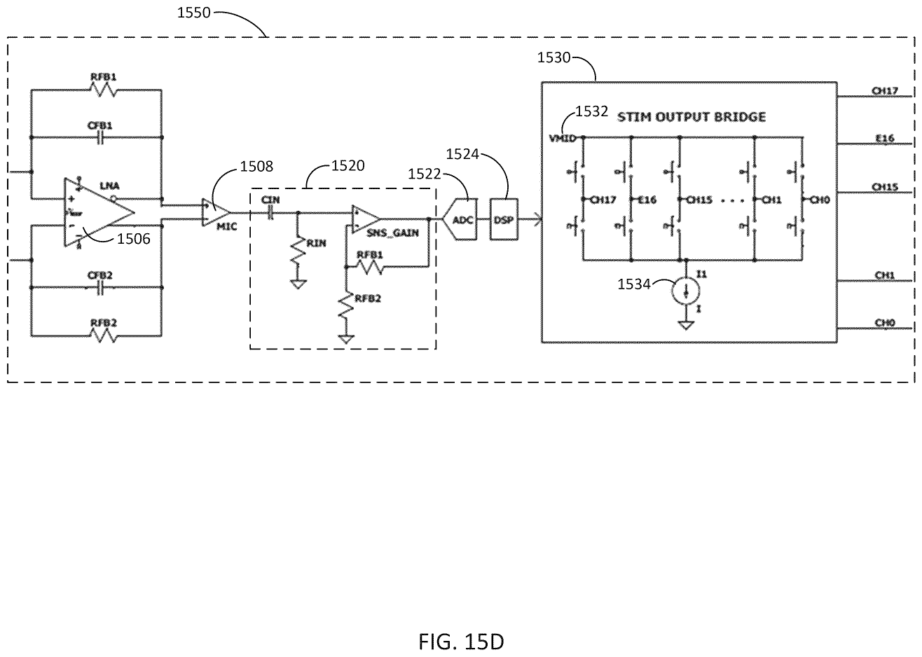

FIG. 15A shows an example diagram of a sensor assembly 1502 comprising a sensor 1503 (e.g., a piezoelectric middle ear sensor) outputting signal affected by an aggressor signal. In the illustrated example, the sensor 1503 outputs a differential signal having a positive output 1560 and a negative output 1562 via conductors in a lead 1504 toward a housing 1550 that includes a differential amplifier 1506. In some examples, the sensor 1503 can be modeled as a Thevenin equivalent differential source where the Thevenin equivalent voltage output from the sensor is labeled VSNS (shown having value V1), and the Thevenin equivalent output capacitance is represented as two capacitances in series with the equivalent voltage, each with value 2*CSNS, where CSNS is the output capacitance of the sensor. In some examples, output capacitance CSNS can be approximately 600 pF. In the illustrated example, positive output and negative output are provided to inputs of differential amplifier 1506. The positive output can be capacitively coupled to a system ground 1505 by a positive side capacitance, CLP. The negative output can be capacitively coupled to a system ground 1505 by a negative side capacitance CLN. In some examples, CLP is approximately 100 pF and CLN is approximately 100 pF. In some examples, positive and negative side capacitances can be achieved using capacitors or other capacitive components.

In the example of FIG. 15A, an aggressor signal (shown as V2) is illustrated as a voltage relative to ground 1505 capacitively coupled to the positive output 1560 with a high side capacitance CHP, which in some examples, is approximately 10 pF, and to the negative output 1562 with a low side capacitance of (CHN+CHNdelta), where CHN is equal to CHP and CHNdelta is the difference in the capacitive coupling of the aggressor signal to the positive output and to the negative output. Capacitive coupling to the positive and negative outputs can be reflective of capacitive coupling to the conductors of the lead 1504 carrying such signals. In some examples, the difference in capacitive coupling to the positive and negative outputs is between approximately 2 pF and 3 pF. In various examples, CHN and CHP are small compared to CSNS and CHNdelta is small compared to CHN and CHP.

If CHNdelta is zero, then the high side aggressor signal capacitive coupling to the positive output 1560 and the low side aggressor signal capacitive coupling to the negative output 1562 are equal, and any aggressor signal coupled into the sensor output is eliminated by the differential amplifier 1506 prior to subsequent processing. If CHNdelta is non-zero (e.g., approximately 2.2 pF), then the mismatch in capacitive coupling of the aggressor signal between the positive and negative outputs leads to an imbalance of the amount of aggressor signal in the positive 1560 and negative 1562 outputs at the differential amplifier, and the aggressor signal causes noise in the output of the differential amplifier.

FIG. 15B shows a circuit diagram showing the effect of CHNdelta on the mismatched capacitive coupling of the aggressor signal V2. As shown, the aggressor signal couples to both the positive output 1560 and the negative output 1562. Coupling capacitance CHP (e.g., 10 pF) and positive side capacitance to ground CLP (e.g., 100 pF) form a voltage divider providing an output (e.g., 1560) to a first input of the differential amplifier 1506. Coupling capacitance CHN+CHNdelta (e.g., 10 pF+2.2 pF=12.2 pF) and negative side capacitance to ground CLN (e.g., 100 pF) form a voltage divider providing negative output 1562 to a second input of the differential amplifier 1506.

The capacitive coupling mismatch CHNdelta (e.g., 2.2 pF) of the aggressor signal to the positive (1560) and negative (1562) outputs can lead to mismatched contributions of the aggressor signal between the inputs of the differential amplifier 1506, which can lead to aspects of the aggressor signal being amplified and/or subsequently processed. This can produce undesired noise in subsequently processed signals, and which may affect signals perceived by a wearer from electrical stimulation.

In some cases, one or more resistors, capacitors, or other components can be used in combination with such capacitances, for example, to generate a suitable signal across the inputs of the differential amplifier representative of the signal between the positive output 1560 and the negative output 1562 from the sensor. FIG. 15C shows an example network 1510 of electrical components that can be used in section 1500 shown in FIG. 15A. In the illustrated example, C36 can provide the capacitance (e.g., 100 pF) of CLP from FIG. 15A, and C33 can provide the capacitance (e.g., 100 pF) of CLN from FIGS. 15A and 15B. The example of FIG. 15D includes additional feedback resistors RFB1 and RFB2 (e.g., 30 MΩ each) used with differential amplifier 1506 not shown in FIGS. 15A and 15B. It will be appreciated that various feedback circuit components can be used. Further, as shown in FIG. 15C, the output of differential amplifier 1506 is provided to another amplifier 1508 to further condition the signal for subsequent processing. The output of amplifier 1508 can be a single-ended voltage with respect to a system ground 1505.

Various values for the electrical components of the example network of FIG. 15C can be used. In an example implementation: C32=C37=22 nF, C33=C36=C43=100 pF, C29=C38=220 nF, CFB1=CFB2=37 pF, R2=R3=4.7 kΩ, R4=R5=1MΩ, RFB1=RFB2=30 MΩ.

FIG. 15D shows additional circuitry that can be used in some examples to provide electrical stimulation based on signals from the sensor. In the example of FIG. 15D, differential amplifier 1506 can be used in the same way as differential amplifiers in FIGS. 15A-C, and can provide an output to an amplifier 1508 for further amplification. The signal output from amplifier 1508 can go through additional filtering and amplification in a pre-processing stage 1520. Pre-processed signal can be converted to a digital signal via an analog-to-digital converter ADC 1522, and processed via a digital signal processor DSP 1524. In some cases, the DSP 1524 provides one or more processes described herein with respect to a signal processor. For instance, in some examples, the DSP 1524 is programmed with a transfer function and is configured to generate stimulations signals to control stimulation output from a cochlear implant system.

A stimulation output bridge 1530 can be controlled by the DSP 1524, for example, where a plurality of controllable switches (e.g., mechanical switches, transistor switches, or the like) can be opened and closed to selectively provide electrical power to one or more of a plurality of channels (e.g., CH0, CH1 . . . . CH17 to form 18 total channels). In some cases, one or more channels (e.g., the channel labeled E16) can serve as a return such that E16 and one other channel combine to form a current path. In some examples, DSP 1524 processes signals received from ADC 1522 according to a transfer function and outputs a stimulation signal to the stimulation output bridge 1530 to cause the stimulation output bridge to provide electrical stimulation according to the transfer function and the signal received from the ADC 1522. In some examples, a voltage 1532 is provided to a side of one or switches, and a current sink 1534 can draw a desired current through various stimulation channels to provide electrical current flow. In some embodiments, a current source can provide current for stimulation. In some embodiments, each stimulation channel includes its own current sink for causing electrical stimulation.

The stimulation output bridge 1530 can be controlled by the DSP 1524 to provide desired electrical stimulation to one or more cochlear electrodes. In some cases, additional components, such as one or more resistors and/or capacitors, can be included downstream of the stimulation output bridge 1530 to condition signals output from the stimulation output bridge 1530 for stimulating tissue. In some cases, a signal output from the stimulation output bridge 1530 is provided to a particular contact electrode, such as a contact electrode on a cochlear electrode (e.g., 116 in FIG. 1) according to the configuration of the stimulation output bridge 1530. In some embodiments, one or more channels are electrically connected to electrodes positioned elsewhere, such as on a device housing (e.g., electrode 530 in FIGS. 5A and 5B). In some embodiments, each channel of the stimulation output bridge can be routed to a given contact electrode to provide stimulation current, returning to one or more return electrodes.

In some examples, components such as the electrical network 1510, differential amplifier 1506 and respective feedback components, pre-processing stage 1520, the ADC 1522, the DSP 1524, and the stimulation output bridge 1530 can be included in a single housing 1550, such as the housing shown and described with respect to FIGS. 5A and 5B. In some cases, an aggressor signal can capacitively couple into the housing 1550, such as at or near a header of the housing (e.g., header 522 and/or 524 in FIG. 5A).

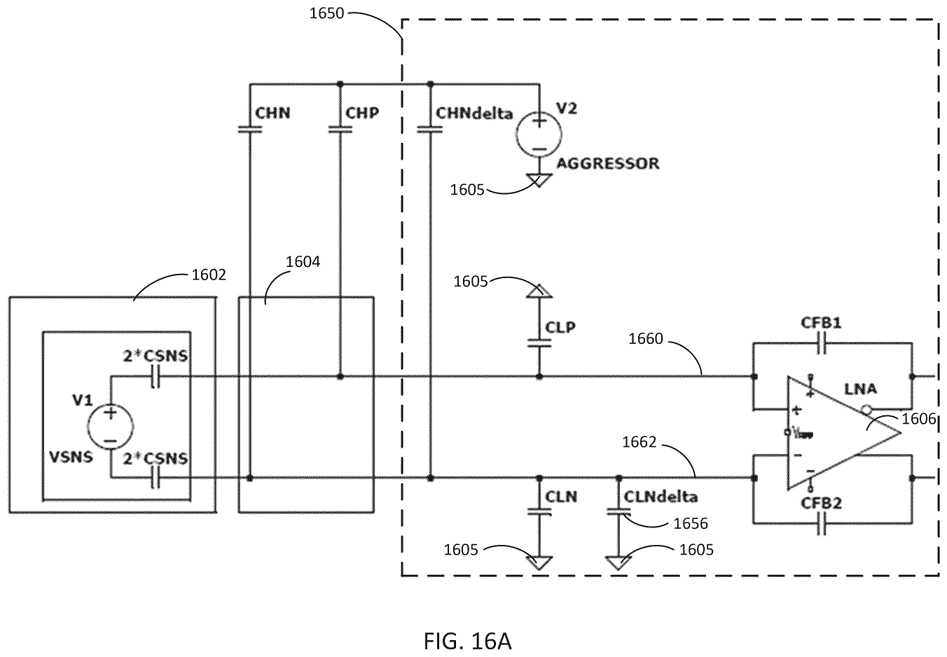

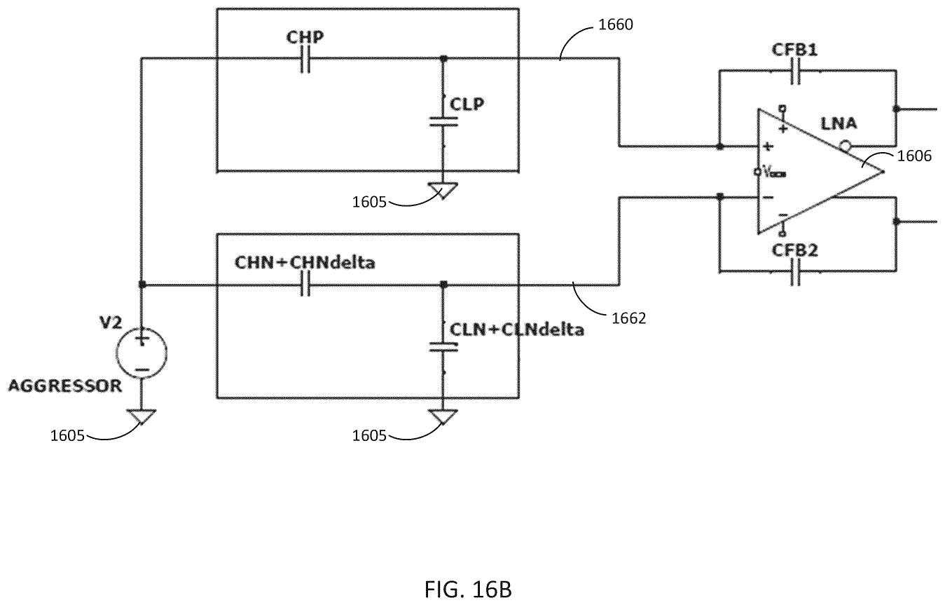

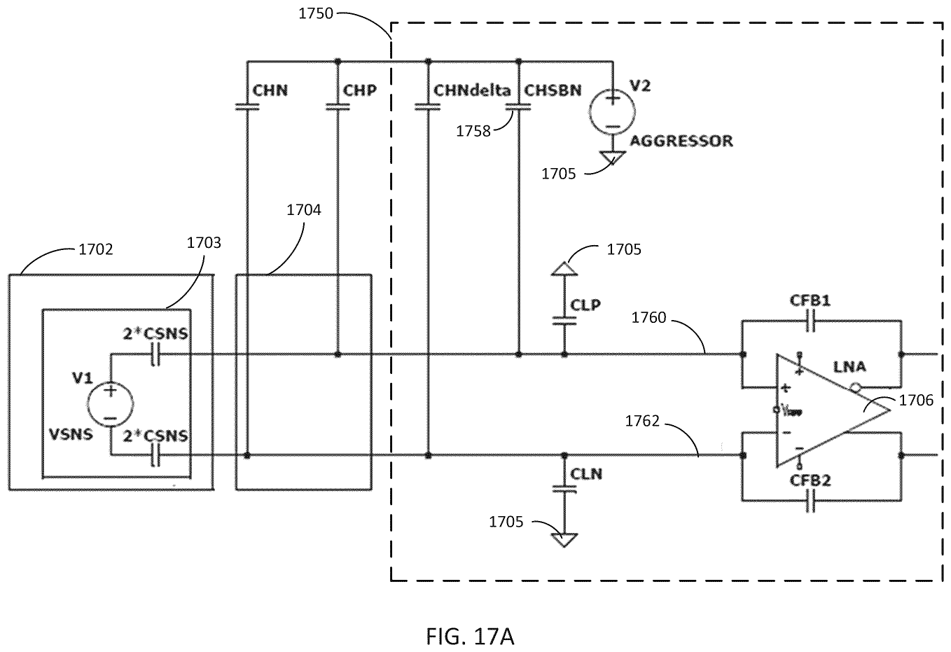

FIG. 16A shows a low side balanced network approach to addressing the mismatched capacitive coupling of the aggressor signal. In a low side balanced approach, a capacitance between a conductor of a lead carrying sensor outputs and system ground can be modified to help balance the difference in the capacitive coupling of the aggressor signal to the positive and negative outputs from the sensor. Extra capacitance can be added between the one of the negative or positive sensor output and ground to help reduce extra charge on the corresponding conductor and balance the capacitive coupling of the aggressor signal to the positive and negative outputs.

FIG. 16A shows an example low side balanced network approach. In the example of FIG. 16A, a sensor assembly 1602 comprising a sensor 1603 (e.g., a piezoelectric middle ear sensor) outputs a positive output 1660 and a negative output 1662 via conductors of a lead 1604, which couples the output to a housing 1650 comprising a differential amplifier 1606. A positive side capacitance CLP is between the positive output and ground 1605 and a negative side capacitance CLN is between the negative output and ground 1605.

Similar to as described with respect to the example of FIG. 15A, an aggressor signal (V2) capacitively couples to the positive output 1660 by a high side coupling capacitance CHP and to the negative output 1662 by a low side coupling capacitance CHN+CHNdelta. As discussed elsewhere herein, this unbalanced capacitance between the capacitive coupling to the positive output 1660 and the negative output 1662 can cause signal artifacts. In the example of FIG. 16A, additional low side balancing capacitance CLNdelta (1656) is in line between the negative output 1662 and ground 1605 to help balance the capacitive coupling of the aggressor signal to the positive and negative sensor outputs. In some cases, CLNdelta is large compared to CHNdelta.

In an example embodiment, CHN and CHP are 10 pF, CHNdelta is 2.2 pF, CLP is 100 pF, and CLN is 100 pF, and CLNdelta is 50 pF. As shown, CLN and CLNdelta are in parallel between the negative output and ground 1605, making the total capacitance 150 pF between the negative output and ground 1605, and 100 pF between the positive output 1660 and ground 1605. In some embodiments, CLNdelta is incorporated as a separate capacitor. In other examples, a value of CLNdelta can be used to choose a new capacitor for use in place of CLN having a capacitance of CLN+CLNdelta. For instance in some embodiments, the negative side capacitance CLN is equal to the positive side capacitance CLP, and the negative side capacitance CLN and the additional negative side capacitance CLNdelta are provided by a single capacitor (e.g., in an example, CLP is provided by a 100 pF capacitor and CLN and CLNdelta are provided by a single 150 pF capacitor).

FIG. 16B shows a circuit diagram showing the effect of CLNdelta on the mismatched capacitive coupling of the aggressor signal. As shown, the aggressor signal couples to both the positive output and the negative output. High side capacitive coupling CHP to the aggressor signal and positive side capacitance CLP form a high side voltage divider, and the low side capacitive coupling (CHN+CHNdelta) to the aggressor signal and the low side capacitance (CLN+CLNdelta) form a low side voltage divider.

As discussed above, CHNdelta can represent the difference in capacitive coupling of the aggressor signal between the positive output and negative output of the sensor signal. The value of CLNdelta can be selected so that the low side voltage divider (including CHNdelta and CLNdelta) has an approximately similar voltage dividing behavior as the high side voltage divider. For instance, in an example embodiment, CHP=CHN=10 pF, CHNdelta=2.2 pF, CLP=CLN=100 pF, and CLNdelta=50 pF. Other values can be used to appropriately balance the difference in capacitive coupling of the aggressor signal between the high and low sides. In some examples, the value of CLNdelta is chosen to approximately balance the behavior of the behavior of the voltage dividers in FIG. 16B at least over a desired range of frequencies while accounting for other system components that affect the voltage dividers (e.g., feedback capacitors of differential amplifier 1606). Balanced capacitive coupling of the aggressor signal to the high and low sides can help introduce the aggressor signal to both inputs of differential amplifier 1606 equally, so that the aggressor signal can be reduced or eliminated at the amplifier 1606.

As shown in FIG. 15C, other circuitry components can be present in a circuit configuration involving CLP and CLN (and CLNdelta). In some cases, such components (e.g., resistors and capacitors, such as shown in FIG. 15C) can give rise to a frequency dependence of the capacitive behaviors of the high and low sides. For instance, in some cases, a given CLNdelta value may effectively balance the capacitive coupling of the aggressor signal to reduce differences at the differential amplifier 1606 from such signals within a given frequency range. However, in some cases, the same capacitance values may be less effective at balancing the capacitance for frequencies outside of such a range. Additionally, in some examples, the gain of the differential amplifier 1606 can affect the optimal balance point (e.g., an optimal CLNdelta value).

In some embodiments, a suitable value for CLNdelta can be determined by measuring or otherwise determining a value of CHNdelta. In some examples, various values of CLNdelta can be tested (e.g., by measuring an effect of aggressor V2 on the output of the differential amplifier) to determine a suitable value for CLNdelta to balance the capacitive coupling of the aggressor signal between the high and low sides. In some examples, such determining can be performed for one or more frequencies or frequency ranges. In some embodiments, CLNdelta (or a single capacitor accounting for CLN+CLNdelta) comprises an adjustable capacitor such that the total capacitance between the negative output and ground 1605 is adjustable without replacing hardware.

While FIGS. 16A and 16B show a larger capacitive coupling of the aggressor signal to the negative sensor output 1660 compared to the positive sensor output 1662, in some examples, the capacitive coupling of the aggressor signal to the positive sensor output 1660 is higher compared to the capacitive coupling of the aggressor signal to the negative sensor output 1662, meaning that the value of CHNdelta in FIG. 16A is effectively negative. In some such examples, a balancing capacitance is incorporated between the positive sensor output 1660 and ground 1605 to supplement capacitance CLP and balance the capacitive coupling of the aggressor signal to the conductors carrying both the positive and negative sensor outputs.

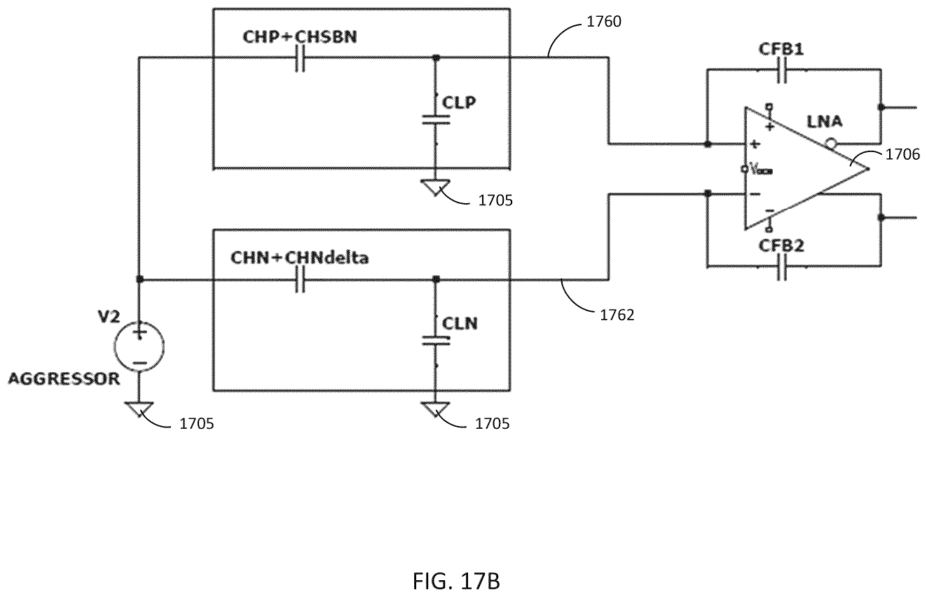

FIG. 17A shows a high side balanced network approach to addressing the aggressor signal. A high side balanced network approach can include modifying a capacitance between the aggressor signal and a conductor of a lead for carrying positive or negative outputs from the sensor.

In the example of FIG. 17A, a sensor assembly 1702 comprising a sensor 1703 (e.g., a piezoelectric middle ear sensor) outputs a positive output 1760 and a negative output 1762 via conductors of a lead 1704, which couples the output to a housing 1750 comprising a differential amplifier 1706. A positive side capacitance CLP is between the positive output 1760 and ground 1705 and a negative side capacitance CLN is between the negative output 1762 and ground 1705.

An aggressor signal V2 is capacitively coupled to positive output 1760 by capacitance CHP and to negative output 1762 by capacitance CHN+CHNdelta, where CHNdelta is the difference in capacitive coupling of the aggressor signal between the positive and negative outputs. In a high side balanced approach, a high side balancing capacitance (CHSBN) (1758) can be included either between the positive output 1760 and the aggressor or between the negative output 1762 and the aggressor to help balance the difference in the capacitive coupling of the aggressor signal to the positive output 1760 and negative output 1762. Including the high side balancing capacitance (CHSBN) can include positioning a capacitance (e.g., a capacitor) between a device header (where the aggressor signal may couple into the device) and the positive output or the negative output (e.g., to the conductor carrying the positive output or the negative output). Additional high side capacitance can be added between the aggressor and the positive output 1760 when the capacitive coupling between the aggressor and the negative output 1762 is larger than the capacitive coupling between the aggressor signal and the positive output 1760. Similarly, a balancing high side capacitance can be added between the aggressor and the negative output 1762 when the capacitive coupling between the aggressor and the negative output 1762 is smaller than the capacitive coupling between the aggressor signal and the positive output 1760.

FIG. 17A shows an example high side balanced network approach, with high side balancing capacitance CHSBN between the aggressor signal and the positive output 1760. In some examples, CHNdelta and CHSBN are small compared to CHN and CHP, CHN and CHP are smaller than sensor output capacitances 2*CSNS. In the some examples, capacitances between the positive output 1760 and ground 1705 (CLP) and between the negative output 1762 and ground 1705 (CLN) are 100 pF, the difference between the capacitive coupling of the aggressor signal to the positive and negative outputs (shown as additional capacitance CHNdelta to the negative output 1762) is approximately 2.2 pF, added balancing capacitance CHSBN is approximately 2.2 pF coupled between the aggressor signal and the positive output 1760 to balance CHNdelta, and the differentially-modeled sensor output capacitances (2*CSNS) are approximately 1200 pF. The balanced CHNdelta (parasitic) and CHSBN (added capacitance) values can balance the capacitive coupling of the aggressor signal to both the positive output 1760 and negative output 1762 as the signal from the sensor 1702 is received at a differential amplifier 1706.

FIG. 17B shows a circuit diagram showing the effect of CHNdelta on the mismatched capacitive coupling of the aggressor signal and balancing the effect with CHSBN. As shown, the aggressor signal couples to both the positive output and the negative output. Negative side capacitive coupling to the aggressor signal (CHN+CHNdelta) and negative side capacitance CLN form a negative side voltage divider, and positive side capacitive coupling to the aggressor signal (modified by added balancing capacitance CHSBN; CHP+CHSBN) and positive side capacitance CLP form a positive side voltage divider.

As discussed above, CHNdelta can represent the physical difference in capacitive coupling of the aggressor signal between the positive output and negative output from the sensor 1702. The value of CHSBN can be selected and intentionally incorporated so that the negative side voltage divider has an approximately similar behavior as the positive side voltage divider with respect to the aggressor signal, resulting in a balanced contribution of aggressor signal in the positive and negative sensor outputs received at the differential amplifier 1706.

The high side balancing capacitance CHSBN can be selected to approximately match the difference in capacitive coupling of the aggressor signal to the positive and negative sides (CHNdelta). In some cases, a value for CHNdelta can be measured or otherwise determined, and CHSBN can be selected to match or approximately match CHNdelta. CHSBN can include a capacitor having an appropriate value or can be achieved by adjusting or implementing an appropriate capacitance in other ways, such as by putting an intentional trace capacitance in a circuit design (e.g., in a printed circuit board trace between components) and/or by adjusting spacings between components, such as one or more components in or around the header, to adjust a capacitance.

In some embodiments, an adjustable capacitor can be used. In some embodiments, the high side balancing capacitance can be implemented using a feedthrough for connecting the positive output 1760 from the sensor 1703 to the housing 1750. For instance, in an example embodiment, a feedthrough in a header of housing 1750 receives a lead carrying positive signal 1760. A high side balancing capacitance CHSBN can be implemented between the feedthrough receiving the positive output 1760 and the housing 1750 (e.g., the header of the housing). In some examples, a dielectric material can be positioned between the feedthrough and the housing (e.g., the header or other aspect of the housing that receives the aggressor signal). The dielectric can be configured (e.g., with dimension and permittivity) to provide a high side balancing capacitance CHSBN between a location of aggressor coupling and a feedthrough receiving the conductor carrying the positive output 1760.

In some cases, the capacitance of CHNdelta is known, and the capacitance of CHSBN is chosen to equal or approximately equal the capacitance of CHNdelta. In some embodiments, different values of CHSBN can be tested with respect to a contribution of the aggressor signal detected in the output of the differential amplifier 1706, and a suitable CHSBN can be determined. For instance, in an example process, only the aggressor signal is present on the positive and negative side conductors of lead 1704 and the sensor 1702 is not itself outputting any signal. CHSBN can be selected so that the output of the differential amplifier 1706 (due only to the aggressor signal) is below a threshold value. In some examples, the capacitance of CHSBN is within approximately +/−30% of the capacitance of CHNdelta. In some examples, (CHP+CHSBN) is within approximately +/−30% of (CHN+CHNdelta).

The high side balancing capacitance CHSBN can optimize the capacitive balance independent of the gain of the differential amplifier. Additionally, other present circuitry (e.g., resistors and capacitors in FIG. 15C) does not change the balancing effects of CHSBN as a function of frequency. CHSBN can effectively balance CHNdelta to offset differences in the capacitive coupling of the aggressor signal to the sensor signal positive and negative outputs across all frequencies.

In some embodiments, the differential amplifier of FIGS. 16A, 16B, 17A, 17B can provide an output to one or more circuit components such as those illustrated in FIGS. 15C and 15D. For instance, in some embodiments, such an output can be amplified, filtered, and processed to provide selective electrical stimulation to a wearer's cochlear tissue.

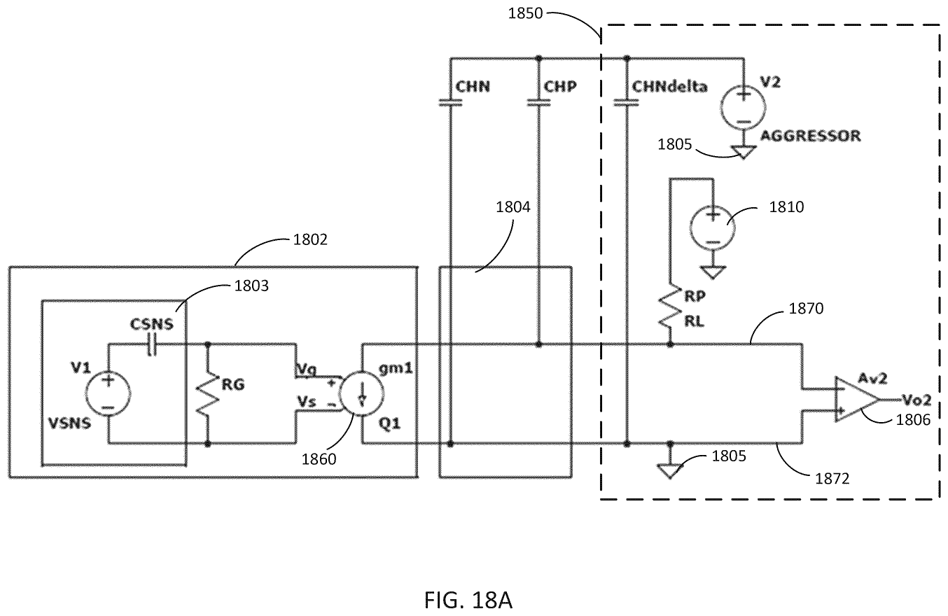

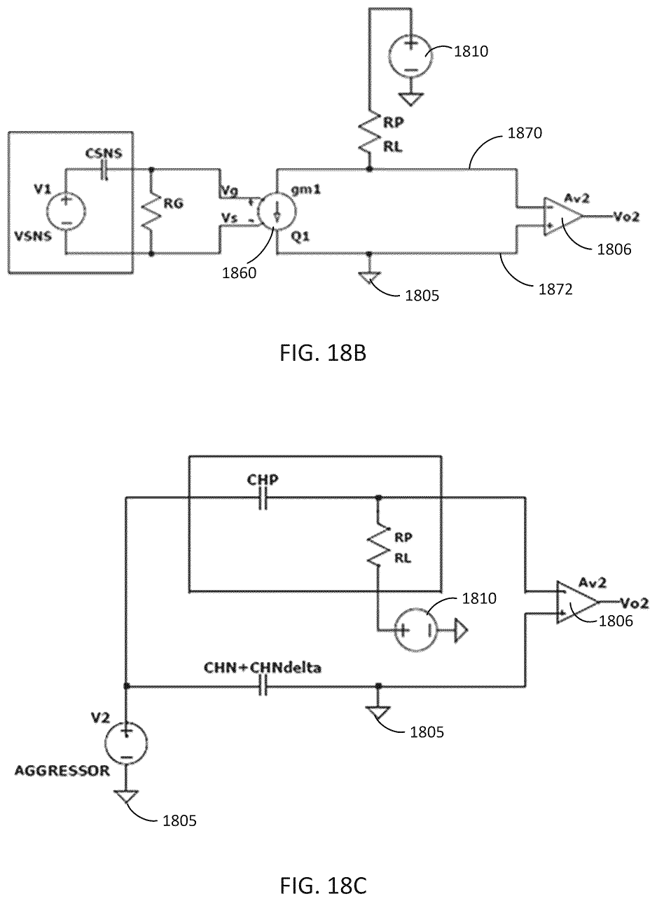

As described elsewhere herein, in some examples, a pre-amplifier (e.g., 716 in FIG. 7) can be used to amplify a signal received from an input source such as a sensor. While, in some examples, a pre-amplifier can be included in a signal processor housing, in some embodiment a pre-amplifier can be included in a sensor housing. FIG. 18A shows an example configuration of a sensor assembly 1802 having a sensor 1803 (e.g., a piezoelectric middle ear sensor) and a pre-amplifier 1860 with gain gm1. In the illustrated example, sensor 1803 outputs a signal that can be amplified by a pre-amplifier 1860, which outputs a positive amplified signal 1870 and a negative amplified signal 1872 via conductors of a lead 1804 to a housing 1850 comprising a differential amplifier 1806. In some embodiments, the pre-amplifier 1860 receives power from amplifier voltage 1810 through a resistor RP. In some examples, amplifier voltage 1810 is approximately 2.5 V. In various examples, housing 1850 can include one or more components similar to those shown in FIG. 15D for amplifying and processing a signal from the sensor and generating and providing electrical stimulation.

In the example of FIG. 18A, an aggressor signal couples to the positive amplified signal 1870 and the negative amplified signal 1872. Similar to discussed elsewhere herein, capacitive coupling of the aggressor signal may be unequal between the positive amplified signal 1870 and the negative amplified signal 1872. In the illustrated example, the aggressor capacitively couples to the positive amplified signal 1870 by a capacitance CHP (e.g., 10 pF), and capacitively couples to the positive amplified signal 1872 by a capacitance CHN+CHNdelta (e.g., 10 pF+2.2 pF). In some examples, sensor 1083 has an output capacitance CSNS approximately equal to 600 pF.

In the illustrated example, the pre-amplifier 1860 is single-ended, with the negative amplified signal 1872 being grounded (at 1805) and the positive amplified signal being separated from an amplifier voltage 1810 by resistance RP having a value of RL. The amplifier voltage 1810 can be used to provide power to the pre-amplifier 1860 through resistor RP. The positive amplified signal 1870 and the (grounded) negative amplified signal 1860 are provided to a differential amplifier 1806.

FIG. 18B shows an illustration of the single-ended amplifier amplifying a signal from the sensor 1803 for providing to the differential amplifier 1806 without the aggressor signal. FIG. 18C shows an illustration of the aggressor signal coupling to the positive and negative amplified signals, with the negative amplified signal 1872 being grounded.

In some cases, pre-amplification of the sensor signal prior to the coupling of the aggressor signal can reduce the proportion of the aggressor signal in the signal to be amplified. However, in some cases, when one side of the amplified signal (the negative amplified signal in FIGS. 18A-C), the mismatched contribution of the aggressor signal between the positive and negative amplified signals is magnified, since the aggressor signal is shunted to ground on one side and couples into the other side.

In an example small signal model of the pre-amplifier 1860 in FIG. 18B, the output from the differential amplifier Vo2=Av2×Vo1, where Av2 is the amplification of the differential amplifier 1806 and Vo1 is the voltage difference between the positive amplified signal 1870 and negative amplified signal 1872 from the sensor 1803 (e.g., the positive amplified signal 1870 when the negative amplified signal 1872 is grounded). In the illustrated example of FIG. 18B, where the negative amplified signal 1872 is grounded, Vo1=−gm1×Vgs×RL, where gm1 is a pre-amplifier transconductance and Vgs is a voltage across the pre-amplifier gate and source terminals. Accordingly, in an example embodiment, Vo2=−gm1×RL×Av2×Vgs, and the overall gain of the pre-amplifier 1860 and the differential amplifier 1806 relative to a voltage input to the pre-amplifier 1860 is Vo2/Vgs=−gm1×RL×Av2.

In FIG. 18C, the aggressor signal capacitively couples to the positive amplified signal and the (grounded) negative amplified signal provided to the differential amplifier 1806. In the case of only aggressor coupling (e.g., no signal from the sensor 1803 and pre-amplifier 1860), the output of the differential amplifier 1806 due to the aggressor signal is Av2×Vo1. If the frequency of the aggressor signal is small (e.g., f<<1/(2×pi×RL×CHP)), then:

Vo2≈Av2×(−2×pi×f×RL×CHP)×Aggressor, and the in-band aggressor gain is

$$Vo 2 / Aggressor \approx Av 2 \times ( - 2 \times pi \times f \times RL \times CHP ) .$$

In some cases, if a frequency f>>1/(2×pi×RL×CHP), then Vo2/Aggressor≈1, where the output of the differential amplifier 1806 is approximately equal to the aggressor signal.

In some embodiments, the contribution of the aggressor signal in a pre-amplified signal is reduced by using a differential pre-amplifier.

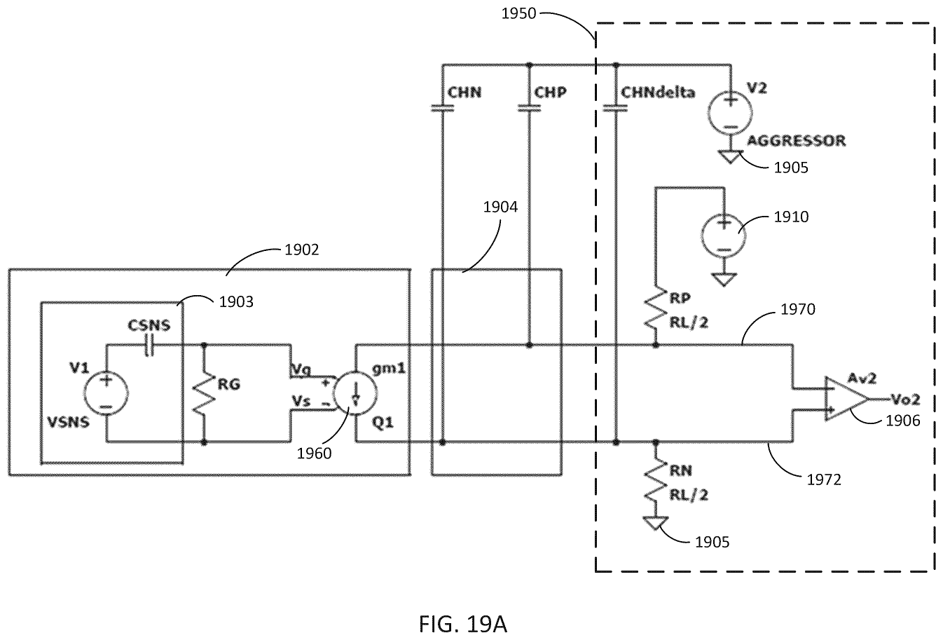

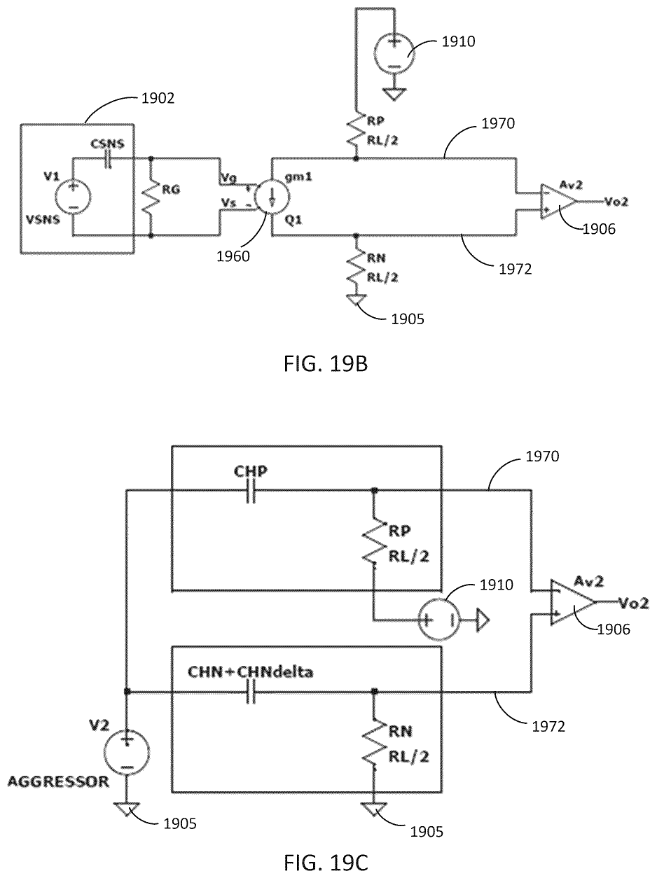

FIG. 19A shows an example configuration of a sensor assembly 1902 comprising a sensor 1903 (e.g., a piezoelectric middle ear sensor) and a differential pre-amplifier 1960 with gain gm1. In the illustrated example, sensor outputs a signal that can be amplified by the pre-amplifier 1960, which outputs a positive amplified signal 1970 and a negative amplified signal 1972. Positive and negative amplified signals can be output from a sensor 1903 within a sensor assembly 1902 via conductors of a lead 1904 to a housing 1950, which can include a differential amplifier 1906. In various examples, housing 1950 can include one or more components similar to those shown in FIG. 15D for amplifying and processing a signal from the sensor and generating and providing electrical stimulation.

In the example of FIG. 19A, an aggressor signal couples to the positive amplified signal and the negative amplified signal. Similar to discussed elsewhere herein, capacitive coupling of the aggressor signal may be unequal between the positive amplified signal 1970 and the negative amplified signal 1972. In the illustrated example, the aggressor capacitively couples to the positive amplified signal by a capacitance CHP (e.g., 10 pF), and capacitively couples to the positive amplified signal by a capacitance CHN+CHNdelta (e.g., 10 pF+2.2 pF).

In the illustrated example, the pre-amplifier 1960 is a differential amplifier, the negative amplified signal 1972 is separated from ground 1905 by resistance RN providing half of a load resistance (RL/2), and the positive amplified signal is separated from an amplifier voltage 1910 (e.g., approximately 2.5 V) by resistance RP providing half of a load resistance (RL/2) for the amplification. In an example differential pre-amplifier embodiment, RL and RP in FIG. 19A have half of the resistance of a resistor RP in a single-ended pre-amplifier embodiment such as in FIG. 18A. The differential pre-amplifier 1960 can receive power from the amplifier voltage 1910 through resistor RP. In the example of FIG. 19A, the positive amplified signal 1970 and the negative amplified signal 1972 are provided to a differential amplifier 1906 with gain Av2.

FIG. 19B shows an illustration of the differential pre-amplifier 1960 amplifying a signal from the sensor for providing to the differential amplifier 1906 without the aggressor signal. FIG. 19C shows an illustration of the aggressor signal coupling to the high- and low-side amplified signals.

In an example small signal model of the pre-amplifier 1960 in FIG. 19B, the output from the differential amplifier Vo2=Av2×(Vo1_N-Vo1_P), where Av2 is the amplification of the differential amplifier 1906, Vo1_P=−gm1×Vgs×RL/2, Vo1_N=gm1×Vgs×RL/2, gm1 is a pre-amplifier transconductance, and Vgs is a voltage across the pre-amplifier gate and source terminals. Vo1_P can be the positive amplified signal 1970 and Vo1_N can be the negative amplified signal 1972. Accordingly, Vo2=−gm1×RL×Av2×Vgs, and the overall gain of the pre-amplifier 1960 and the differential amplifier 1906 relative to a voltage input to the pre-amplifier 1960 is Vo2/Vgs=−gm1×RL×Av2.

In FIG. 19C, the aggressor signal capacitively couples to positive amplified signal and the negative amplified signal provided to the differential amplifier 1906. In the case of only aggressor coupling (e.g., no signal from the sensor 1903 and pre-amplifier 1960), the output of the differential amplifier 1906 due to the aggressor signal is Av2× (Vo1_P-Vo1_N). If the frequency of the aggressor signal is small (e.g., f<<1/(2×pi×RL/2× (CHN+CHNdelta))), then:

Vo2≈Av2×(−2×pi×f×RL/2×CHNdelta)×Aggressor, and the in-band aggressor gain is

$$Vo 2 / Aggressor \approx Av 2 \times ( - 2 \times pi \times f \times ( RL / 2 ) \times CHNdelta ) .$$

Compared to the single-sided pre-amplification embodiment, the common-mode output impedance of the differential pre-amplification is reduced by a factor of 2 (RP=RN=RL/2). Further, the aggressor signal couples more evenly to the positive and negative amplified signals compared to the single-sided pre-amplification embodiment, where the negative amplified signal is grounded. In the example of FIG. 18A, the capacitive mismatch of the aggressor signal coupling to the positive amplified output 1870 and negative amplified output 1872 is 10 pF (since the negative amplified signal is grounded). In the example of FIG. 19A, the capacitive mismatch is only 2.2 pF of capacitive difference between the positive and negative amplified outputs. Accordingly, the in-band aggressor gain is reduced by approximately a factor of four in the illustrated embodiments (CHP=10 pF capacitive difference in the single-ended pre-amplifier vs. CHNdelta=2.2 pF capacitive difference in the differential pre-amplifier).

Accordingly, since the common-mode output impedance and capacitive coupling differential between the positive and negative amplified signals factor into the in-band aggressor gain, the in-band aggressor gain is reduced by approximately a factor of 8 (approximately −18 dB): a factor of two (−6 dB) in the common-mode output impedance and approximately a factor of four (−12 dB) in the capacitive coupling difference between the positive and negative amplified outputs.

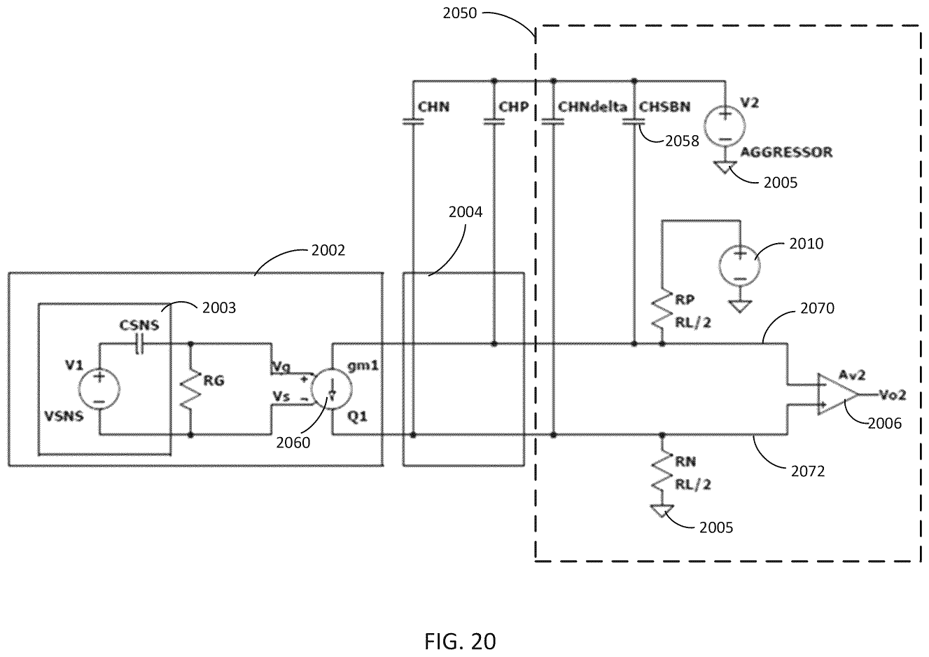

In various embodiments, pre-amplification and capacitance balancing described herein can be combined. FIG. 20 shows an example embodiment combining a differential pre-amplification and high side capacitive balancing. FIG. 20A shows an example configuration of a sensor assembly 2002 comprising a sensor 2003 (e.g., a piezoelectric middle ear sensor) and a differential pre-amplifier 2060 with transconductance of gm1. In the illustrated example, sensor outputs a signal that can be amplified by the pre-amplifier 2060, which outputs a positive amplified signal 2070 and a negative amplified signal 2072. Positive and negative amplified signals can be output from a sensor assembly via conductors of a lead 2004 to a housing 2050, which can include a differential amplifier 2006. In various examples, housing 2050 can include one or more components similar to those shown in FIG. 15D for amplifying and processing a signal from the sensor and generating and providing electrical stimulation.

In the example of FIG. 20, an aggressor signal couples to the positive amplified signal 2070 and the negative amplified signal 2072. Similar to discussed elsewhere herein, capacitive coupling of the aggressor signal may be unequal between the positive amplified signal 2070 and the negative amplified signal 2072. In the illustrated example, the aggressor capacitively couples to the positive amplified signal 2070 by a capacitance CHP (e.g., 10 pF), and capacitively couples to the positive amplified signal 2072 by a capacitance CHN+CHNdelta (e.g., 10 pF+2.2 pF), where CHNdelta represents the difference in capacitive coupling between the aggressor and the positive and negative amplified signals.

Similar to as described with respect to the differential pre-amplifier in FIGS. 19A-C, the differential pre-amplifier 2060 of FIG. 20 can amplify the signal from the sensor so that the contribution of the sensor signal to the input to differential amplifier Av2 is comparatively larger than that of the aggressor signal. Differential pre-amplifier can be powered by amplifier voltage 2010 (e.g., approximately 2.5 V) via resistance RP. The differential pre-amplifier 2060 (with both positive and negative amplified signals being separated from ground 2005 by a resistance of RL/2) reduces the common-mode output impedance compared to a single-ended pre-amplifier (e.g., 1860 in FIG. 8A) as described elsewhere herein. Additionally, a non-grounded negative amplified signal results in the aggressor signal capacitively coupling to the low side and not being shunted to ground. Accordingly, the aggressor signal in FIG. 20 capacitively couples to the positive amplified signal 2070 at CHP (10 pF in FIG. 20) and to the negative amplified signal 2072 at CHN+CHNdelta (10 pF+2.2 pF in FIG. 20), with CHNdelta representing the difference in capacitive coupling between the aggressor and the positive and negative signals.

Similar to as described above, for example, with respect to FIGS. 17A and 17B, a high side balancing capacitance CHSBN (2058) that approximately matches the CHNdelta capacitance difference can be incorporated into the system between the aggressor and the positive amplified signal. The high side balancing capacitance can include, for example, a capacitor having capacitance CHSBN positioned between a header of housing 2050 and a line carrying the positive amplified signal 2070. The high side balancing capacitance can be used to balance the coupling capacitance of the aggressor to the positive and negative amplified signals.

As described above with respect to FIGS. 19A-C, in some cases with a differential pre-amplifier, the in-band aggressor gain can be approximated by

$$Vo 2 / Aggressor \approx Av 2 \times ( - 2 \times pi \times f \times ( RL / 2 ) \times CHNdelta )$$

However, as noted, CHNdelta is the difference in capacitive coupling between the positive and negative amplified signal. When a high side balancing capacitance CHSBN approximating the value of CHNdelta is placed as shown in FIG. 20, the difference in capacitive coupling between the positive and negative amplified signal decreases, and accordingly, the CHNdelta term in the approximation for in-band aggressor gain decreases. When the high side balancing capacitance CHSBN equals the difference in natural capacitive coupling between the positive and negative amplified signals (e.g., when CHSBN=CHNdelta), the in-band aggressor gain approximation goes to zero. Thus, in some such embodiments, the signal from the sensor can be amplified by the differential pre-amplifier, and the aggressor signal can be equally capacitively coupled to both the high and low amplified signals by incorporating a balancing capacitance CHSBN so that the aggressor signal can be eliminated at the differential amplifier (e.g., the in-band aggressor gain goes to zero).

Various example embodiments describe ways of addressing a mismatch of capacitive coupling of an aggressor signal between positive and negative amplified sensor outputs. Some labels used in the various illustrative drawings, even if represented using the same label, need not have equivalent values from figure to figure, embodiment to embodiment, or implementation to implementation. For instance, values of capacitances CHN and CHP (and CHNdelta) can vary among systems, and/or may change within a system. Signals such as Vo2 shown in different figures need not be the same signal from figure to figure. For example, Vo2 from amplifier 2006 in FIG. 20 is not necessarily the same as Vo2 from amplifier 1906 in FIGS. 19A-C. Similarly, values such as the aggressor signal value V2, sensor voltage V1, transconductance gm1, gain Av2, capacitances CLP, CLN, amplified voltages Vo1_N, and Vo1_P need not be equal between illustrated examples.

Additional low side balancing capacitance CLNdelta (1656 in FIG. 16A) can be selected in a low side balancing approach to have an appropriate value to balance capacitive coupling of an aggressor signal to the positive (e.g., 1660) and negative (e.g., 1662) outputs. In some examples, additional low side balancing capacitance CLNdelta can be chosen based on the values of capacitances CHP, CLP, CHN, CLN, and CHNdelta in a given implementation to balance capacitive coupling over desired frequency ranges. A high side balancing capacitance CHSBN (e.g., 1758 in FIG. 17A) can be selected in a high side balancing approach to have an appropriate value to balance capacitive coupling of an aggressor signal to positive (e.g., 1760) and negative (e.g., 1762) outputs. In some examples, high side balancing capacitance CHSBN (e.g., 1758 in FIG. 17A) can be chosen based on the values of capacitances CHP, CLP, CHN, CLN, and CHNdelta in a given implementation to balance capacitive coupling over desired frequency ranges.

In some embodiments, the cochlear implant system is configured to predict and compensate for stimulation-induced noise in the sensor signal path by modeling the noise as a sum of basis functions, each associated with a respective contact electrode of a stimulation array. For instance, in some examples, each basis function can represent an impulse response of the system to stimulation at a given electrode, and the overall noise at any given time can be modeled as the sum of the noise contributions from each electrode being used for stimulation.

This approach leverages the fact that the system has knowledge of the stimulation commands delivered to each electrode over time, allowing for accurate prediction of the resulting noise artifact.

The shape and amplitude of each electrode's basis function can be determined as a function of stimulation level output from that electrode (e.g., a voltage provided at the contact electrode). In some examples, a desired stimulation is based on a level of electrical current applied to tissue, so the current amplitude and stimulation path impedance (e.g., tissue impedance and tissue/electrode interface impedance) can be related to the stimulation noise. For instance, in embodiments where stimulation is current-based, the stimulation path impedance can be used to determine the contact electrode voltage to drive stimulation. Accordingly, in some examples, the noise artifact at a given contact electrode can be based on the stimulation path impedance associated with that electrode.

In some embodiments, the basis functions for each contact electrode can be measured or otherwise determined to calibrate the basis function for each contact electrode. For instance, in some examples, stimulation noise (e.g., represented as an aggressor signal) can be measured as a function of stimulation current magnitude for a plurality of current magnitudes for each of a plurality of contact electrodes. The results of the calibration can be stored in a lookup table, for example, indexed by stimulation current and/or stimulation path impedance. This lookup table may be updated or recalibrated over time to account for changes in stimulation path impedance. The lookup table information can represent, for example, a basis function for each electrode that estimates a contribution of each electrode to stimulation noise as a function of current provided by that electrode.

In some embodiments, during operation, a cochlear implant system can be configured to monitor stimulation commands delivered to each electrode (e.g., an amount of current to be used for stimulation) and, for each time frame (e.g., a stimulation frame such as described herein), predict the expected noise artifact caused by stimulation by summing the expected contribution to stimulation noise for each electrode. The system can be configured to subtract the predicted noise from the sensor signal in real time or near real time to improve the fidelity of the sensed signal and reduce the impact of stimulation artifacts on downstream processing.

In some embodiments, the system may drive stimulation using individual electrodes while in a quiet environment and while measuring the sensor signal. If the environment is quiet such that there is little or no acoustic stimulus causing an output from the sensor (e.g., a piezoelectric middle ear sensor), the input received at the signal processor will generally be due to the aggressor signal caused by the stimulation artifacts. By analyzing the resulting data, the system can determine the resulting aggressor signal due to stimulation from a given electrode. In some embodiments, a system can be configured to provide stimulation from each electrode individually and in a quiet environment in order to determine an aggressor signal associated with the stimulation via each electrode.

In some cases, the aggressor signal for each electrode can be a function of stimulation parameters. Various stimulation parameters that can affect the aggressor signal include stimulation current, stimulation impedance (e.g., stimulation path impedance including tissue impedance, electrode impedance, and electrode/tissue interface impedance), pulse width, interphase time, number of and/or set of enabled vs. disabled electrodes, interconnection of electrodes (e.g., whether electrodes are arranged in parallel, such as using multiple return electrodes), and type(s) of source element(s) (e.g., source and/or sink) for providing stimulation. For instance, in an example implementation, a return electrode can be set at an intermediate DC potential while one or more other electrodes are connected to a source or sink in order to provide biphasic stimulation relative to the return electrode. Various such electrode configurations and other stimulation parameters can contribute to aspects of the aggressor signal present during stimulation.

In some examples, a corresponding artifact can be measured for each of a plurality of values for one or more stimulation parameters for each of a plurality of contact electrodes. In an example embodiment, a stimulation artifact aggressor signal can be measured for a plurality of stimulation current values for each of a plurality of contact electrodes. The aggressor signals associated with stimulation from each particular electrode can form basis functions for an overall aggressor signal associated with stimulation from a plurality of electrodes during a stimulation frame.

In some embodiments, in addition or as an alternative to identifying basis functions using stimulation from each electrode individually, in some embodiments, a cochlear implant system can use random or pseudo-random stimulation patterns, for which the stimulation output is known, to output stimulation in an otherwise quiet environment. In the quiet environment, signals received at the signal processor will generally represent stimulation noise. By analyzing the resulting signals sensed at the signal processor in view of the known stimulation pattern(s) using multiple electrodes, the system (e.g., via the signal processor) can be configured to back-calculate the basis functions for each electrode (e.g., using multi-linear regression or similar techniques). In various examples, a multi-linear regression for calculating basis functions can be solved using traditional calculation methods (e.g., using matrix inversion) or by alternate approaches, for example, by progressively diagonalizing a multi-linear regression as new data is acquired. Progressive diagonalization can act to continuously calibrate the basis functions as data is acquired, and the system can quickly adapt to changes that occur over time such as changes in tissue impedance, etc.

Empirical determination of basis functions (e.g., using single-electrode stimulation or random or pseudo-random stimulation patterns) allows the system to adapt to individual patient anatomy, electrode placement, and system-level variations. In some embodiments, a lookup table including the basis functions can be stored in a memory in communication with one or more processing components of the signal processor.

By modeling the stimulation-induced noise as a sum of electrode-specific basis functions and compensating the sensor signal accordingly, the system provides accurate, adaptive, and patient-specific artifact rejection. This enhances the fidelity of the sensor signal, improves overall system performance, and may lead to better patient outcomes in cochlear implant applications.

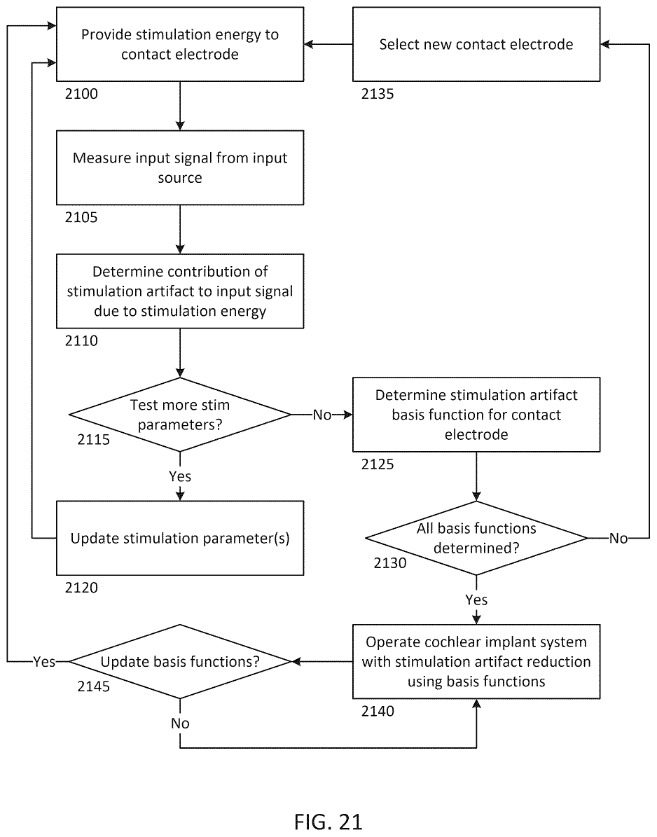

FIG. 21 shows an example process flow diagram for establishing basis functions for one or more contact electrodes. In the example of FIG. 21, the process includes providing stimulation energy to a contact electrode (2100), measuring an input signal from the input source (2105), and determining a contribution of a stimulation artifact (e.g., an aggressor signal such as described herein) to the input signal due to the stimulation energy (2110). In some examples, the stimulation energy is a voltage provided to the contact electrode whether current is provided via the electrode. Additionally or alternatively, stimulation energy can include a prescribed current to be sourced or sunk via the contact electrode. In some examples, the stimulation energy is provided in 2100 in a quiet environment such that the measured input signal at 2105 primarily or exclusively includes stimulation artifacts.

The process of FIG. 21 further includes, if more stimulation parameters are to be tested for establishing a basis function (as indicated at 2115), updating a stimulation parameter (e.g., a current or voltage applied to the contact electrode) and repeating steps 2100, 2105, and 2110. The process includes, if all desired stimulation parameters for establishing a basis function have been tested (e.g., a desired plurality of current or voltage values associated with the electrode), determining a stimulation artifact basis function for the contact electrode (2125). In some examples, the basis function for the contact electrode includes an amount of aggressor signal (e.g., voltage) picked up at the signal processor due to stimulation energy provided to that contact electrode, and can be a function of one or more stimulation parameters (e.g., current or voltage).

The process of FIG. 21 continues with a determination of whether all basis functions have been determined (2130). If not, a new contact electrode is selected (2135), and the basis function determination process can be performed for that electrode. If all basis functions have been determined, the process continues with operating a cochlear implant system with stimulation artifact reduction using the basis functions (2140). In various embodiments, basis functions can be determined for any number of contact electrodes and can be used in systems comprising any number of contact electrodes.

As noted elsewhere herein, in some cases, basis functions can be determined by applying stimulation patterns and back-calculating individual basis function. In some examples, stimulation energy can be provided to a plurality of contact electrodes within a stimulation frame and an input signal from the input source can be measured. This can be repeated for various combinations of stimulation parameters and/or contact electrodes in order to provide sufficient data for a system to back-calculate basis functions for individual electrodes (e.g., using multi-linear regression or similar techniques).

As described elsewhere herein, in some embodiments, the basis functions can be updated, for example, to account for changing stimulation path impedance. The process can include determining whether to update the basis functions (2145). If not, the cochlear implant system can continue to operate with stimulation artifact reduction using basis functions (2140). In various examples, basis functions updates can be initiated automatically, such as according to a predetermined schedule, or manually, for example, in response to a user or clinician command to calibrate stimulation interference correction in response to perceived noise.

In some examples, one or more steps of the process of FIG. 21 can be performed via an implantable signal processor of a cochlear implant system. Additionally or alternatively, one or more steps can be performed via an external computing device. In some cases, one or more steps may be performed manually or at the direction of a system user (e.g., updating basis functions).

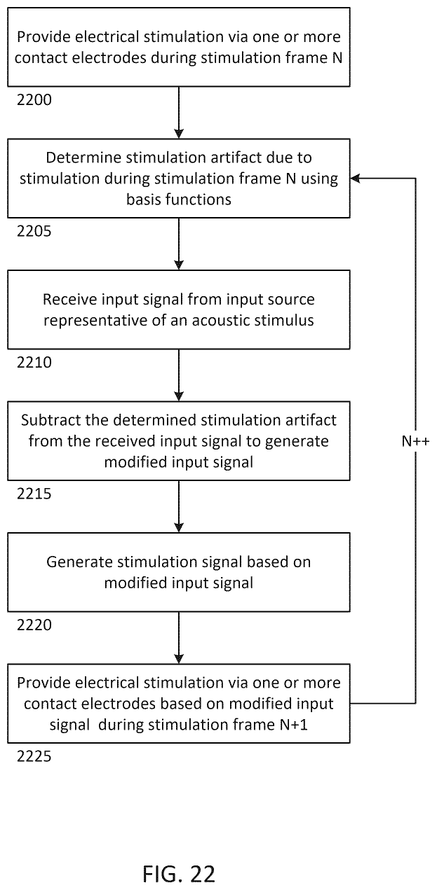

Once basis functions are established, the basis functions can be used to reduce stimulation artifact reduction during cochlear implant system operation. FIG. 22 shows an example process flow diagram illustrating operation of a cochlear implant system reducing stimulation artifacts using basis functions.

The example of FIG. 22 includes providing electrical stimulation via one or more contact electrodes during stimulation frame N (2200). The stimulation can be based on an input signal received from an input source and a signal processor transfer function such as described elsewhere herein. The process further includes determining an artifact due to stimulation during the stimulation frame N using the basis functions (2205).

In an example embodiment each of a plurality of 16 electrodes has an associated basis function f₁, f₂, . . . , f₁₆. As described, such basis functions can be functions of, for example, voltage or current provided during a stimulation frame. When stimulation is provided during a stimulation frame, a stimulation artifact (e.g., an aggressor signal) can be approximated as a linear combination of the basis functions: A₁f₁+A₂f₂+ . . . +A₁₆f₁₆ where Aᵢ are coefficients, which can be related to an amount of current or voltage provided from each respective electrode during the stimulation frame. In some embodiments, the basis functions fᵢ are functions of the voltage and/or current, and the corresponding coefficient for a given stimulation frame is a 1 or a 0 depending on whether the corresponding electrode is active during a stimulation frame. In some cases, all coefficients Aᵢ are effectively 1, with the effects of the stimulation parameters (e.g., current and/or voltage) and whether or not the electrode is active during a stimulation frame being accounted for by the basis function itself.

The process of FIG. 22 further includes receiving an input signal from an input source representative of an acoustic stimulus (2210) and subtracting the determined stimulation artifact from the received input signal to generate a modified input signal (2215). In some examples, the received input signal in 2210 corresponds to data received during or approximately during the stimulation frame N.

The process further includes generating a stimulation signal based on the modified input signal (2220) and providing electrical stimulation via one or more contact electrodes based on the modified input signal during stimulation frame N+1 (2225). The electrical stimulation can be based on a stimulation signal that is based on the modified input signal and a transfer function of the signal processor.

The process can continue with the value of N incremented such that the electrical stimulation during stimulation frame N+1 in step 2225 serves as the stimulation frame for step 2205 in a subsequent iteration for which an updated stimulation artifact can be determined and subtracted from a next received input signal. In some examples, basis functions associated with stimulation during a particular stimulation frame are used for artifact reduction in the immediately following stimulation frame. In other examples, basis functions associated with stimulation during a particular stimulation frame are used for artifact reduction in a later stimulation frame, but not necessarily the immediate next stimulation frame.

Processes such as that of FIG. 22 can iteratively repeat such that ongoing stimulation can be used to predict resulting stimulation artifacts using established basis functions, and such resulting stimulation artifacts can be reduced or eliminated. In some embodiments, the process of FIG. 22 is executed within an implantable signal processor of a cochlear implant system.

In various examples, signal analysis and processing to determine a stimulation artifact using basis functions and/or subtracting the determined stimulation artifact can be performed in the time domain or in the frequency domain.

In some cases, predictive compensation of stimulation-induced noise may be performed in real time, with the system continuously monitoring output stimulation and modifying sensor signals using combinations of basis functions. In some embodiments, as each new stimulation event occurs, the system can be configured to retrieve corresponding basis function(s) from a lookup table and determine a predicted stimulation interference signal. The sum of all electrode contributions to the stimulation interference can be subtracted from the raw sensor signal, yielding a compensated signal with reduced artifact content.

The described approach provides several advantages over traditional artifact rejection methods. By leveraging knowledge of the stimulation history and electrode-specific impulse responses, the system can achieve more precise and adaptive artifact compensation. The use of lookup tables and data-driven calibration can allow the system to accommodate manufacturing variations, patient-specific anatomy, and long-term changes in electrode impedance or system configuration.

Basis function modeling and compensation technique described herein enables the cochlear implant system to predict and remove stimulation-induced artifacts with high accuracy and adaptability. This results in improved signal fidelity, enhanced system robustness, and the potential for better auditory outcomes for implant recipients.

As described elsewhere herein, in some examples, filtering processes can be used to reduce or eliminate stimulation artifacts. In some embodiments, filters can be used in combination with capacitive coupling balancing and/or amplification solutions.

In some cases, a filter can be configured to attenuate a narrow band of signal around one or more frequencies (e.g., a filter frequency and, in some cases, one or more harmonics). Various processes can be used to determine the frequency or frequencies to attenuate. In some examples, a stimulation frame rate and/or one or more harmonics thereof can be attenuated based on a known stimulation frame rate. In some examples, frequencies related to stimulation artifact noise are attenuated based on a measured or estimated interference.

Although the range of frequencies attenuated to reduce the noise due to a stimulation artifact can be narrow, in some cases, attenuating portions of the input signal can lead to an attenuation of useful signal. In some embodiments, one or more frequencies near those attenuated can be amplified in order to help compensate for the useful signal that is attenuated.

For instance, in an example embodiment, a stimulation artifact has a peak amplitude at approximately 781 Hz, and a narrow filter attenuates the input signal in a narrow frequency band around the artifact frequency of approximately 780.5-781.5 Hz. Other attenuation ranges are possible. The signal processor can be configured to amplify the input signal outside of the attenuated range on one or both sides of the attenuated range (e.g., from approximately 730-780 Hz and/or from approximately 782-832 Hz). In embodiments where a predetermined range of frequencies are amplified around an attenuated frequency or range of frequencies, various frequency range sizes can be used.

In some embodiments, the signal processor can be configured to identify the frequency or frequencies attenuated by addressing the noise due to stimulation artifacts and amplify frequencies on one or both sides of the attenuated frequencies. In some examples, amplifying such frequencies can be performed as parts of methods described herein, such as, for example, in generating modified input signals (e.g., step 950 in FIG. 9) and/or updating a transfer function (e.g., in step 1040 in FIG. 10) to reduce noise due to stimulation artifacts.

In some cases, the noise due to the stimulation artifact has much narrower frequency content compared to typical acoustic environments. Filtering a narrow amount of input signal around the stimulation artifact frequency can also attenuate a small portion of the acoustic environment within the input signal. However, amplifying nearby frequencies outside of the attenuated range can help to restore lost signal amplitude from a broad-spectrum scene. For instance, if the source of a sound in an acoustic environment is not a pure tone sound, attenuating signals in a narrow frequency band while amplifying signals near but outside of that band may result in an imperceivable or acceptable change in perceived sound while also attenuating the stimulation interference.

As described elsewhere herein. In some embodiments, a signal processor is configured to divide an input signal received from an input source (e.g., a middle ear sensor) into a plurality of frequency bands, and electrical stimulation at individual contact electrodes can be provided for a given frequency band. If one or more frequencies or ranges of frequencies within one of the plurality of frequency bands are attenuated to reduce noise due to stimulation artifacts, the signal processor can be configured to amplify one or more frequencies near the attenuated frequency or frequencies and within the same frequency band of the plurality of frequency bands. This can help preserve the overall amplitude in that frequency band and its corresponding stimulation despite the attenuation within the band to reduce the noise due to stimulation artifacts.

In an example embodiment, a signal processor is configured to filter an input signal using a known filter configured to attenuate one or more frequencies. For example, the signal processor can know the stimulation frame rate for stimulation and be programmed with a filter (e.g., a comb filter or a notch filter) to attenuate one or more frequencies associated with the frame rate, such as the frame rate itself and/or one or more harmonics thereof.

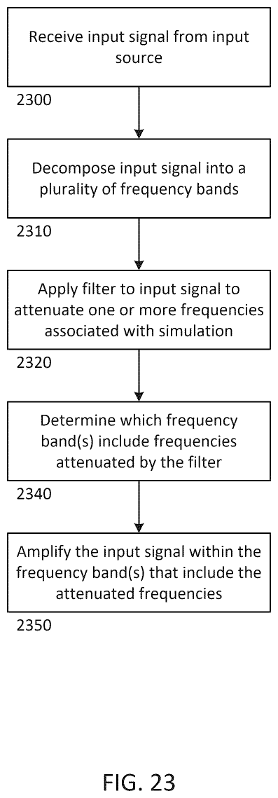

FIG. 23 shows a process flow diagram illustrating an example process of attenuating one or more frequencies from an input signal and amplifying portions of the input signal to compensate for signal lost by the attenuation. The example process of FIG. 23 includes receiving an input signal from an input source (2300) and decomposing the input signal into a plurality of frequency bands (2310). The frequency bands can correspond to different frequency channels for stimulation, for example, where a unique contact electrode is used to provide electrical stimulation to nearby cochlear tissue corresponding to portion of the input signal in a corresponding frequency band.

The process includes applying a filter to the input signal to attenuate one or more frequencies associated with electrical stimulation (2320). In some embodiments, such a filter attenuates one or more frequencies associated with a stimulation frame rate. In some examples, the filter attenuates the stimulation frame rate within the input signal. Additionally or alternatively, the filter can attenuate one or more harmonics of the stimulation frame rate. The signal processor can be configured to perform the filtering process (e.g., via a digital filter) based on a programmed frame rate and/or programmed filter properties. As described elsewhere herein, such filtering can reduce or eliminate stimulation artifacts in the input signal.

The process of FIG. 23 further includes determining which frequency band(s) include the one or more frequencies attenuated by the filter (2340). For example, if the filter attenuates that stimulation frame rate frequency, step 2340 can include determining which frequency band the stimulation frame rate frequency is in. The process further includes amplifying the input signal within the frequency band(s) that include the attenuated frequency or frequencies (2350). In some embodiments, amplifying the input signal within a frequency band comprises applying a uniform amplification across all frequencies within the frequency band. In some embodiments, amplifying the frequencies within such a frequency band comprises amplifying the frequencies by an amount such that the overall intensity of the input signal within that frequency band (e.g., an integral of amplitude over frequency) is the same as if no attenuation had occurred in step 2320.

As described, filtering the input signal can include attenuating one or more frequencies. In some cases, the filter includes a notch filter attenuating a single frequency (e.g., the stimulation frame rate) or narrow range of frequencies. In some cases, the filter includes a comb filter with several notches attenuating several single frequencies or narrow ranges of frequencies. One or more frequency bands into which the input signal is decomposed can include one or more frequencies or narrow ranges of frequencies attenuated by the filtering in step 2320.

In various examples, steps of the process in FIG. 23 can be omitted or permuted. For instance, while the decomposing the input signal into frequency bands (2310) is shown as being before the applying the filter to the input signal (2320), in some examples, the filtering can be performed prior to decomposing the input signal. Further, in some cases, the one or more attenuated frequencies of the filtering and the frequency bands into which the input signal is decomposed can be predetermined (e.g., based on a programmed signal processor transfer function and known stimulation frame rate), and steps 2310, 2320, 2340, and 2350 can be performed in any order.

The various systems and methods described herein can reduce the effects of interference on the signals of the system. Reducing interference can improve the system for use by a wearer, as reducing interference reduces unintended modification or change to a received input signal and manifesting in unintended effects in the associated electrical stimulation.

Various solutions described herein can be used alone or in combination to address interference due to stimulation artifacts.

Various examples have been described. These and others are within the scope of this disclosure.

1. A cochlear implant system comprising:
an input source configured to receive an acoustic stimulus and generate an input signal representative of the acoustic stimulus;
a cochlear electrode comprising a plurality of contact electrodes;
a stimulator in communication with the cochlear electrode and configured to provide electrical stimulation to cochlear tissue via the contact electrodes of the cochlear electrode; and
a signal processor in communication with the stimulator and the input source, the signal processor being programmed with a transfer function and being configured to:
receive the input signal from the input source at a signal processor input;
generate a stimulation signal based on the received input signal and a transfer function of the signal processor, the stimulation signal comprising a stimulation pattern having an electrical output corresponding to each of the plurality of contact electrodes; and
determine an amount of interference at the signal processor input associated with the stimulation signal.

2-18. (canceled)

19. The cochlear implant system of claim 1, wherein:
determining the amount of interference at the signal processor input associated with the stimulation signal comprises applying a time domain adaptive filter to the input signal.

20. The cochlear implant system of claim 19, wherein the time domain adaptive filter to the input signal comprises a filter period equal to a stimulation frame, the stimulation frame corresponding to an amount of time it takes to apply electrical stimulation via each of the plurality of contact electrodes.

21. The cochlear implant system of claim 20, wherein applying the time domain adaptive filter to the input signal comprises:
determining a plurality of estimated stimulation artifact values in a corresponding plurality of bins within the filter period; and
subtracting each of the plurality of estimated stimulation artifact values from a corresponding data point of the input signal.

22. The cochlear implant system of claim 21, further comprising updating each of the plurality of estimated stimulation artifact values based on the received input signal to refine the estimated stimulation artifact values.

23. The cochlear implant system of claim 22 wherein updating each of the plurality of estimated stimulation artifact values based on the received input signal comprises updating, in a sequence, the estimated stimulation artifact value of each of the plurality of bins and repeating the sequence.

24. The cochlear implant system of claim 1, wherein determining the amount of interference at the signal processor input associated with the stimulation signal comprises applying a Kalman filter to the input signal based on damped sinusoid mathematical model.

25-27. (canceled)

28. The cochlear implant system of claim 1, wherein the signal processor is configured to attenuate a narrow band of frequencies of the input signal corresponding to a stimulation artifact and amplify one or more frequencies outside of the attenuated band to compensate for attenuation of useful signal.

29. The cochlear implant system of claim 1, wherein the signal processor is configured to:
divide the input signal into a plurality of frequency bands;
attenuate one or more frequencies within a frequency band are attenuated to reduce noise due to stimulation artifacts; and
amplify one or more frequencies within the same frequency band other than the one or more attenuated frequencies.

30. A cochlear implant system comprising:
a signal processor;
a housing containing the signal processor, the housing comprising a header positioned on an external surface of the housing;
a sensor configured to output a sensor signal representative of an acoustic stimulus;
a lead coupling the sensor to the header, the lead comprising a first conductor and a second conductor such that the sensor signal is communicated from the sensor via the first conductor and the second conductor, the sensor signal having a positive sensor signal communicated via the first conductor and a negative sensor signal communicated via the second conductor; and
a high side balancing capacitance electrically coupled between the header of the housing and either the first conductor of the lead or the second conductor of the lead.

31. The cochlear implant system of claim 30, wherein the high side balancing capacitance comprises a balancing capacitor electrically coupled between the header of the housing and the first conductor of the lead.

32. The cochlear implant system of claim 30, further comprising a positive side capacitance electrically coupled between the first conductor and a reference voltage and a negative side capacitance electrically coupled between the second conductor and the reference voltage, the negative side capacitance having the same capacitance as the positive side capacitance.

33. The cochlear implant system of claim 32, wherein the positive side capacitance comprises a positive side capacitor and the negative side capacitance comprises a negative side capacitor.

34. The cochlear implant system of claim 30, wherein the sensor comprises a differential pre-amplifier electrically coupled to the first conductor and the second conductor, the differential pre-amplifier configured to amplify and output a differential signal from the sensor such that the positive sensor signal comprises a positive amplified sensor signal and the negative sensor signal comprises a negative amplified sensor signal.

35. The cochlear implant system of claim 30, wherein the high side balancing capacitance is configured to balance a difference in capacitive coupling of an aggressor signal between the first conductor and the second conductor.

36. The cochlear implant system of claim 35, wherein the high side balancing capacitance comprises a balancing capacitor having a capacitance value selected to substantially match a difference in capacitive coupling of the aggressor signal to the first conductor and the second conductor.

37. The cochlear implant system of claim 36, wherein the high side balancing capacitance is adjustable to allow tuning of the capacitive coupling between the first conductor and the header.

38. The cochlear implant system of claim 35, wherein the high side balancing capacitance is electrically coupled between the header of the housing and the first conductor of the lead and is selected such that a voltage divider formed by the high side balancing capacitance and a positive side capacitance electrically coupled between the first conductor and a reference voltage is substantially matched to a voltage divider formed by a coupling capacitance between the aggressor signal and the second conductor and a negative side capacitance electrically coupled between the second conductor and the reference voltage.

39. The cochlear implant system of claim 30, further comprising a differential amplifier electrically coupled to the first conductor and the second conductor, the differential amplifier configured to amplify a differential signal corresponding to the sensor signal received from the sensor.

40-72. (canceled)
